SEQUENCE LISTING (41 sequences)

SEQ ID NO: 1 - DNA, 20 bp, Artificial Sequence
  NLRP1#1
gatagcccgagtgacatcgg

SEQ ID NO: 2 - DNA, 20 bp, Artificial Sequence
  NLRP1#2
agcccgagtgacatcggtgg

SEQ ID NO: 3 - DNA, 20 bp, Artificial Sequence
  CASP1#1
acagacaagggtgctgaaca

SEQ ID NO: 4 - DNA, 20 bp, Artificial Sequence
  CASP1#2
attgactccgttattccgaa

SEQ ID NO: 5 - DNA, 20 bp, Artificial Sequence
  CASP1#3
tgactccgttattccgaaag

SEQ ID NO: 6 - DNA, 20 bp, Artificial Sequence
  ASC#2
gctaacgtgctgcgcgacat

SEQ ID NO: 7 - DNA, 20 bp, Artificial Sequence
  ASC#3
catgtcgcgcagcacgttag

SEQ ID NO: 8 - DNA, 20 bp, Artificial Sequence
  UBR2#1
tgcataacttgaactttgag

SEQ ID NO: 9 - DNA, 20 bp, Artificial Sequence
  UBR2#2
tccatgcacaaaacacaagt

SEQ ID NO: 10 - DNA, 20 bp, Artificial Sequence
  UBR2#3
actgtggtgatactgaagcc

SEQ ID NO: 11 - DNA, 20 bp, Artificial Sequence
  UBR2#4
tgatactgaagcctggaaag

SEQ ID NO: 12 - DNA, 20 bp, Artificial Sequence
  UBR2#5
ttgcatgctgtttaatgatg

SEQ ID NO: 13 - DNA, 20 bp, Artificial Sequence
  ZER1#5
cagggacccaatcaatcatg

SEQ ID NO: 14 - DNA, 20 bp, Artificial Sequence
  ZER1#6
ggcaggacgagtctatccag

SEQ ID NO: 15 - DNA, 20 bp, Artificial Sequence
  ZER1#7
aggctgaagaagctctcgtg

SEQ ID NO: 16 - DNA, 20 bp, Artificial Sequence
  ZYG11B#5
agcgctcgtaaggatcctcg

SEQ ID NO: 17 - DNA, 20 bp, Artificial Sequence
  ZYG11B#6
gcagtggctttgcaaccatg

SEQ ID NO: 18 - DNA, 20 bp, Artificial Sequence
  ZYG11B#7
cttggttaagttaaatacac

SEQ ID NO: 19 - protein, 53 aa, Artificial Sequence
  Human 3Cpro cleavage site
LMPWIHELPAGCTQGSERRVLRQLPDTSGRRWREISASLLYQALPSSPDHESP

SEQ ID NO: 20 - protein, 53 aa, Artificial Sequence; identical to SEQ ID NO: 19
  Chimpanzee 3Cpro cleavage site

SEQ ID NO: 21 - protein, 53 aa, Artificial Sequence; identical to SEQ ID NO: 19
  Bonobo 3Cpro cleavage site

SEQ ID NO: 22 - protein, 53 aa, Artificial Sequence; identical to SEQ ID NO: 19
  Gorilla 3Cpro cleavage site

SEQ ID NO: 23 - protein, 53 aa, Artificial Sequence
  Orangutan 3Cpro cleavage site
LMPWIHELPAGCTQGSERRVLRQLPDTSGRRWREISSSLLYQALPSSPDYESP

SEQ ID NO: 24 - protein, 53 aa, Artificial Sequence
  Gibbon 3Cpro cleavage site
LMPWICELPAGCTQGSERRVLRQLPDTSGRRWREISSSLLYQALPSSPDHESP

SEQ ID NO: 25 - protein, 53 aa, Artificial Sequence
  Olive Baboon 3Cpro cleavage site
LRPWNRELPGECTQGSERRVLRQLPDTSGRRWREISSSLLYQAFPSSPDFESP

SEQ ID NO: 26 - protein, 53 aa, Artificial Sequence
  Gelada 3Cpro cleavage site
LRPWNRELPAECTQGSERRVLRQLPDTSGRRWREISSSLLYQALPSSPDFESP

SEQ ID NO: 27 - protein, 53 aa, Artificial Sequence
  Sooty Mangabey 3Cpro cleavage site
LRPWNRELPAECTQGSERRVLRQLPDTSGHRWREISSSLLYQAFPSSPDFESP

SEQ ID NO: 28 - protein, 53 aa, Artificial Sequence
  Drill 3Cpro cleavage site
LRPWNRELPAECTQGSERRVLRQLPDTSGRRWREISSSLLYQAFPSSPDFESP

SEQ ID NO: 29 - protein, 53 aa, Artificial Sequence; identical to SEQ ID NO: 26
  Macaque 3Cpro cleavage site

SEQ ID NO: 30 - protein, 53 aa, Artificial Sequence
  Pig-tailed Macaque 3Cpro cleavage site
LRPWNRELPAECTQGSERRVLRQLPDTSGRRWREISSSLLYQVLPSSPDFESP

SEQ ID NO: 31 - protein, 53 aa, Artificial Sequence
  Vervet-AGM 3Cpro cleavage site
LRPWNRELPAECTQGSERRVLRQLPDTSGRRWREISSSFLYQALPSSPDLESP

SEQ ID NO: 32 - protein, 53 aa, Artificial Sequence
  Black Snub-nosed Monkey 3Cpro cleavage sit
LRPWNCDLPAECTQGSERRVLRQLPDTSGHRWREISSSLLYQALPSSPDFESP

SEQ ID NO: 33 - protein, 53 aa, Artificial Sequence
  Golden Snub-nosed Monkey 3Cpro cleavage sit
LRPWNRDLPAECTQGSERRVLRQLPDTSGHRWREISSSLLYQALPSSPDFESP

SEQ ID NO: 34 - protein, 53 aa, Artificial Sequence
  Angola Colobus 3Cpro cleavage site
LRSWNHELPAECTQVSERRVLRQLPDTSGHRWREISSSLLYQALPSSPDFESP

SEQ ID NO: 35 - protein, 52 aa, Artificial Sequence
  Capuchin 3Cpro cleavage site
LRSWIPELQGCTQGSERSILRHLPDTSGHHWREITSSLLYQAPLSSPDYGSP

SEQ ID NO: 36 - protein, 52 aa, Artificial Sequence
  Bolivian Squirrel Monkey 3Cpro cleavage site
LRSWIPELQGCTQGSERSVLRHLPDMSGHRWREITSSLLYQAPLSSPDYGSP

SEQ ID NO: 37 - protein, 52 aa, Artificial Sequence
  Marmoset 3Cpro cleavage site
LRSQIPELHGCTQRSERSVSRRLPDTSGHQWREITSSLLYQAPLSSPDYGSP

SEQ ID NO: 38 - protein, 52 aa, Artificial Sequence
  Ma′s Night Monkey 3Cpro cleavage site
LRSSIPELQGCTQGSKRSILRHLPDTSGHRWREITSSLLYQAPLSSPDYGSP

SEQ ID NO: 39 - protein, 53 aa, Artificial Sequence
  Tarsier 3Cpro cleavage site
LGILGPGSLESPQDSEKRRILRWPPDTSGNNWREIKPLPLFEALSSFPVYQSP

SEQ ID NO: 40 - protein, 19 aa, Artificial Sequence
  Mouse Lemur 3Cpro cleavage site
SSPDFPCFGLCHPESPRLQ

SEQ ID NO: 41 - protein, 19 aa, Artificial Sequence
  Coquerel′s Sifaka 3Cpro cleavage site
PPPLLPWGTPRSQKREGFQ

(19) United States
(12) Patent Application Publication
(10) Pub. No.: US 2023/0172933 A1
(43) Pub. Date: Jun. 8, 2023

(54) USE OF A COMPOUND OR COMPOSITION COMPRISING AN INHIBITOR OF NLRP1 INFLAMMASOME ACTIVATION FOR THE TREATMENT OF HUMAN AIRWAY INFLAMMATION

(71) Applicants: Nanyang Technological University, Singapore (SG); Agency for Science, Technology and Research, Singapore (SG)

(72) Inventors: Lei ZHONG, Singapore (SG); Bruno REVERSADE, Singapore (SG); Kim Samirah ROBINSON, Singapore (SG)

(21) Appl. No.: 17/912,802

(22) PCT Filed: Mar. 18, 2021
(86) PCT No.: PCT/SG2021/050142

§ 371 Date: Sep. 19, 2022

(30) Foreign Application Priority Data
Mar. 19, 2020 (SG) 10202002526P

(51) Int. Cl. A61K 31/519; A61K 31/7064; A61K 31/428; A61K 38/06; A61K 31/69; A61P 31/16; A61P 11/00

(52) U.S. Cl. CPC A61K 31/519; A61K 31/7064; A61K 31/428; A61K 38/06; A61K 31/69; A61P 31/16; A61P 11/00

(57) ABSTRACT

The present invention provides a method of prophylaxis or treatment of airway inflammation and/or related complications triggered by Enterovirus 3C protease-activated NLRP1, a compound or composition comprising said compound for use in the method, and use of said compounds in medicament preparation for the prophylaxis or treatment of human airway inflammation and/or related complications triggered by Enterovirus 3C protease-activated NLRP1.

### FIELD OF THE INVENTION

The present invention provides a method of prophylaxis or treatment of airway inflammation and/or related complications triggered by Enterovirus 3C protease-activated NLRP1, a compound or composition comprising said compound for use in the method, and use of said compounds in medicament preparation for the prophylaxis or treatment of human airway inflammation and/or related complications triggered by Enterovirus 3C protease-activated NLRP1.

### BACKGROUND OF THE INVENTION

The human innate immune system employs a multitude of germline-encoded sensor proteins to detect microbial infections and kickstart the first-line immune response (Jones et al., Science 354 (2016)). Nod-like receptor (NLR) proteins are a family of innate immune sensors that can detect pathogen-associated molecular patterns (PAMPs) in the cytosol [Jones et al., Science 354 (2016); Shaw et al., Curr. Opin. Immunol. 20: 377-382 (2008); Kanneganti et al., Immunity 27: 549-559 (2007)]. Upon activation, NLR proteins nucleate the assembly of inflammasome complexes, leading to pyroptotic cell death and secretion of pro-inflammatory cytokines, such as IL-1β and IL-18 [Latz, Current Opinion in Immunology 22: 28-33 (2010)]. Among all human NLR sensors, NLRP1 remains one of the few whose cognate PAMP ligands have not been identified. Germline activating mutations in NLRP1 cause Mendelian syndromes characterized by multiple self-healing keratoacanthomas of the skin and hyperkeratosis in the laryngeal and corneal epithelia [Grandemange et al., Annals of the Rheumatic Diseases 76: 1191-1198 (2017); Zhong et al., Cell 67: 187-202 (2016); Mamai et al., J. Invest. Dermatol. 135: 304-308 (2015)]. Carriers of certain common NLRP1 single nucleotide polymorphisms (SNPs) experience increased risks for auto-immune diseases such as asthma and vitiligo [Sui et al., Arthritis Rheum. 64: 647-654 (2012); Levandowski et al., PNAS 110: 2952-2956 (2013)]. Human NLRP1 differs from its rodent homologues in terms of domain organization, ligand specificity and tissue distribution [Sand et al., Cell Death Dis 9: 24 (2018)] and its exact role in human immune response in vivo is still unclear.

Recent studies have delineated a novel pathway by which certain alleles of the rodent NLRP1 homolog, Nlrp1b, are activated by bacterial toxins, such as the anthrax lethal factor (LF) [Boyden and Dietrich, Nat. Genet. 38: 240-244 (2006); Newman et al., PLoS Pathog. 6: e1000906 (2010); Hellmich et al., PLoS One 7: e49741 (2012); Levinsohn et al. PLoS Pathog. 8: e1002638 (2012); Chavarria-Smith et al., PLoS Pathog. 9: e1003452 (2013)]. Anthrax LF directly cleaves Nlrp1b close to its N-terminus [Levinsohn et al. PLoS Pathog. 8: e1002638 (2012); Chavarria-Smith et al., PLoS Pathog. 9: e1003452 (2013)]. This cleavage causes N-degron-mediated degradation of the auto-inhibitory N-terminal fragment, thus freeing the non-covalently bound FIIND^{UPA}-CARD (a.a.1213-1474) fragment to activate caspase-1 [Chui et al., Science 364: 82-85 (2019); Sandstrom et al., Science 364 (2019); Xu et al. EMBO J. (2019)](FIG. 1A). The consensus LF cleavage site is conspicuously absent in human NLRP1, which contains a N-terminal PYRIN domain not found in rodents (FIG. 1A). As such, LF does not cleave or activate human NLRP1. Despite their differences, both human NLRP1 and rodent homologs can be activated by chemical inhibitors of dipeptidases DPP8 and DPP9, although the underlying mechanisms remain to be fully elucidated [Zhong et al., J. Biol. Chem. 293: 18864-18878 (2018); Okondo et al., Cell Chem Biol. 25: 262-267 (2018); de Vasconcelos et al., Life Sci Alliance 2 (2019)]. Rodent Nlrp1 can also be activated by Toxoplasma gondii infection in a process that does appear to involve protease-mediated cleavage [Cirelli et al., PLoS Pathog. 10: e1003927 (2014); Ewald et al., Infect. Immun. 82: 460-468 (2014)]. It remains an open question as to whether any naturally occurring pathogen- or danger-associated signals (DAMPs) can activate human NLRP1, and whether they do so via a similar ‘functional degradation’ pathway as documented in mice. There is a need to identify possible cognate PAMP ligand(s) that trigger the human NLRP1 inflammasome and to assess the mechanisms by which they activate NLRP1 in order to ameliorate human airway inflammation.

### SUMMARY OF THE INVENTION

The present invention arises from the identification of a PAMP that can trigger the NLRP1-activated inflammasome in human airways. More particularly, the inventors have identified the NLRP1-activated inflammasome pathway, involving cullin^{ZER1/ZYG11B} and NEDD8-activating enzyme (NAE), and potential inhibitors of same.

In a first aspect of the invention there is provided a compound or composition comprising said compound for use in the prophylaxis or treatment of human airway inflammation and/or related complications triggered by Enterovirus 3C protease-cleaved NLRP1, wherein the compound or composition is an inhibitor of NLRP1 inflammasome activation.

The Enterovirus genus encompasses 234 human pathogens that form 7 species spread worldwide: human enteroviruses A through D (HEV-A, HEV-B, HEV-C, and HEV-D) and human rhinoviruses A through C (HRV-A, HRV-B, and HRV-C). Echoviruses and coxsackievirus B (CV-B) are classified within the HEV-B species, and polioviruses (PVs) are classified within HEV-C.

In some embodiments, the said 3C protease is from a virus species selected from the group comprising human enterovirus A (HEV-A), human enterovirus B (HEV-B), human enterovirus C (HEV-C), human enterovirus D (HEV-D), human rhinovirus A (HRV-A), human rhinovirus B (HRV-B), and human rhinovirus C (HRV-C).

Cleavage of NLRP1 by the 3C protease results in a polypeptide fragment of NLRP1 having an N-terminal Glycine which is susceptible to an N-glycine degron pathway.

In some embodiments, said compound inhibits N-glycine degron pathway ubiquitination and degradation of NLRP1 cleavage products.

In some embodiments, the compound inhibits cullin^{ZER1/ZYG11B}.

In some embodiments, the compound is an inhibitor of NEDD8-activating enzyme (NAE).

In some embodiments, the compound or composition comprises pharmaceutically acceptable salts or solvates, or pharmaceutically functional derivatives of said inhibitor compound.

In some embodiments, the compound is selected from the group comprising:
MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof;
TAS4464, IUPAC name 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-, or hydrochloride salt thereof;
ZM223, IUPAC name N-[6-[[2-(4-aminophenyl)sulfanylacetyl]amino]-1,3-benzothiazol-2-yl]-4-(trifluoromethyl)benzamide;
MG132, IUPAC Name: benzyl N-[(2S)-4-methyl-1-[[(2S)-4-methyl-1-[[(2S)-4-methyl-1-oxopentan-2-yl]amino]-1-oxopentan-2-yl]amino]-1-oxopentan-2-yl]carbamate, and
Bortezomib, IUPAC Name: [(1R)-3-methyl-1-[[(2S)-3-phenyl-2-(pyrazine-2-carbonyl amino)propanoyl]amino]butyl]boronic acid.

In some embodiments, the 3C protease is from a human rhinovirus.

In some embodiments, the composition comprises an inhibitor compound with a pharmaceutically-acceptable adjuvant, diluent or carrier.

In a second aspect of the invention, there is provided a use of a compound or composition according to any aspect of the invention for the manufacture of a medicament for the prophylaxis or treatment of human airway inflammation and/or related complications triggered by Enterovirus 3C protease-activated NLRP1.

In some embodiments, the medicament reduces IL-13 and IL-18 secretion, ASC oligomerization and/or lytic cell death.

In some embodiments, said 3C protease is from a virus species selected from the group comprising human enterovirus A (HEV-A), human enterovirus B (HEV-B), human enterovirus C (HEV-C), human enterovirus D (HEV-D), human rhinovirus A (HRV-A), human rhinovirus B (HRV-B), and human rhinovirus C (HRV-C).

In some embodiments, said compound inhibits N-glycine degron pathway ubiquitination and degradation of NLRP1 cleavage products.

In some embodiments, said compound inhibits cullin^{ZER1/ZYG11B}.

In some embodiments, said compound is an inhibitor of NEDD8-activating enzyme (NAE).

In some embodiments, the compound is selected from the group comprising:

MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof;

TAS4464, IUPAC name 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-, or hydrochloride salt thereof;

ZM223, IUPAC name N-[6-[[2-(4-aminophenyl)sulfanylacetyl]amino]-1,3-benzothiazol-2-yl]-4-(trifluoromethyl)benzamide;

MG132, IUPAC Name: benzyl N-[(2S)-4-methyl-1-[[(2S)-4-methyl-1-[[(2S)-4-methyl-1-oxopentan-2-yl]amino]-1-oxopentan-2-yl]amino]-1-oxopentan-2-yl]carbamate, and

Bortezomib, IUPAC Name: [(1R)-3-methyl-1-[[(2S)-3-phenyl-2-(pyrazine-2-carbonyl amino)propanoyl]amino]butyl]boronic acid.

It is within the person skilled in the art's ability to determine effective dose ranges for administration to a subject in need of prophylaxis or treatment of human airway inflammation and/or related complications triggered by Enterovirus 3C protease-activated NLRP1. In some embodiments an effective dose range may be between about 0.1 μM to 1 μM.

In a third aspect of the invention, there is provided a method of prophylaxis or treatment of airway inflammation and/or related complications triggered by Enterovirus 3C protease-activated NLRP1, in a subject, comprising administering a therapeutically effective amount of a compound or composition of any aspect of the invention.

In some embodiments, said 3C protease is from a virus species selected from the group comprising human enterovirus A (HEV-A), human enterovirus B (HEV-B), human enterovirus C (HEV-C), human enterovirus D (HEV-D), human rhinovirus A (HRV-A), human rhinovirus B (HRV-B), and human rhinovirus C (HRV-C).

In some embodiments, said compound inhibits N-glycine degron pathway ubiquitination and degradation of NLRP1 cleavage products.

In some embodiments, said compound inhibits cullin^{ZER1/ZYG11B}.

In some embodiments, said compound is an inhibitor of NEDD8-activating enzyme (NAE).

In some embodiments, the compound is selected from the group comprising:

MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof;

TAS4464, IUPAC name 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-, or hydrochloride salt thereof;

ZM223, IUPAC name N-[6-[[2-(4-aminophenyl)sulfanylacetyl]amino]-1,3-benzothiazol-2-yl]-4-(trifluoromethyl)benzamide;

MG132, IUPAC Name: benzyl N-[(2S)-4-methyl-1-[[(2S)-4-methyl-1-[[(2S)-4-methyl-1-oxopentan-2-yl]amino]-1-oxopentan-2-yl]amino]-1-oxopentan-2-yl]carbamate, and

Bortezomib, IUPAC Name: [(1R)-3-methyl-1-[[(2S)-3-phenyl-2-(pyrazine-2-carbonyl amino)propanoyl]amino]butyl]boronic acid.

In some embodiments, a subject administered said prophylaxis or treatment will have reduced IL-1 secretion, ASC oligomerization and/or lytic cell death in the airway compared to an untreated subject.

In some embodiments, the compound is MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof.

### BRIEF DESCRIPTION OF THE FIGURES

FIG. 1 shows that enteroviral 3Cpros activate the human NLRP1 inflammasome. (A) Domain structures of human NLRP1 and rodent Nlrp1b. Murine Nlrp1b is activated by anthrax lethal factor (LF) toxin cleavage, followed by the proteasomal degradation the auto-inhibitory N-terminal fragment. (B) Percentage of 293T-ASC-GFP-NLRP1-FLAG cells with ASC-GFP specks after over-expression of Myc-tagged viral proteases. Cells were fixed 24 hours after transfection of the indicated proteases or empty vector control. Talabostat (2 μM, 24 hrs) treatment was used as a positive control. The number of cells with ASC-GFP specks were visually scored with wide-field epifluorescence microscopy at 20× magnification. More than 100 cells were scored for ASC-GFP speck formation per condition. n=3 biological replicates. *P<0.05; **P<0.01 (Two-way ANOVA); n.s., not significant. (C) Blue-Native PAGE (BN-PAGE) analysis of NLRP1 self-oligomerization. 293T-NLRP1-FLAG cells were transfected the indicated plasmids. One day after transfection, cells were either mock treated or treated with 2.5 μM MG132 for 24 hrs. Cells were lysed 48 hours after transfection. 20 μg of total lysate was used for Blue-Native PAGE or SDS-PAGE followed by Western blot. (D) The effect of MG132 on NLRP1-dependent ASC-GFP speck formation. 293T-ASC-GFP or 293T-ASC-GFP-NLRP1-FLAG cells were transfected with HRV14-3Cpro and treated with 2.5 μM MG132 6 hours post transfection. ***P<0.001 (Two-way ANOVA). (E) HRV14-3Cpro induces mature IL-1B secretion and ASC oligomerization in immortalized human keratinocytes. Tet-ON HRV-3Cpro or 3Cpro^{C146A} N/TERT keratinocytes and conditioned media were harvested 24 hours after 1 μg/mL doxycycline (DOX). Conditioned media were concentrated 10 times before SDS-PAGE and IL-1β Western blotting. Endogenous ASC oligomers were extracted by 1% SDS after covalent crosslinking of the 1% NP40-insoluble pellets with 1 mM DSS in PBS. (F) Live cell imaging of ASC-GFP N/TERT cells after HRV14-3Cpro transfection. White arrows indicate pyroptotic cells with ASC-GFP specks and PI inclusion. (G) Cas9 control and NLRP1 KO keratinocytes were stably transduced with Tet-ON HRV14-3Cpro. Cells were induced and harvested as in FIG. 1E. *=a non-specific band observed for ASC Western blot. (H) IL-1p ELISA of conditioned media from NLRP1 KO and Cas9 control Tet-ON HRV14-3Cpro N/TERT keratinocytes 24 hours after DOX induction. ***P<0.001; ****P<0.0001 (Two-way ANOVA). n=3 independent cell seedings and inductions. (1) Trypan blue inclusion assay of NLRP1 KO and Cas9 control Tet-ON HRV14-3Cpro N/TERT keratinocytes 24 hours after DOX induction. **P<0.01 (Two-way ANOVA), n=3 independent cell seedings and inductions as in FIG. 1H.

FIG. 2 shows that HRV-3Cpro can act as a potent trigger of pyroptotic cell death in immortalized human keratinocytes. (A) Expression of Myc-tagged viral proteases in 293T-ASC-GFP-NLRP1-FLAG cells. Two exposure times were shown. Note that Myc-HRV16-3Cpro was lowly expressed but could activate ASC-GFP specks. (B) Representative wide-field fluorescent images of ASC-GFP specks, NLRP1-HA and Myc-tagged proteases. 293T-ASC-GFP cells were co-transfected with NLRP1-HA and Myc-tagged gZiPro, HRV14-3Cpro or HRV14-3Cpro^{C146A} and fixed 24 hours post transfection. (C) Validation of ASC KO in N/TERT cells. ASC KO #2 was chosen for further experiments. (D) Validation of CASP1 KO in N/TERT cells. #1 was chosen for further experiment shown in FIG. 2E. (E) N/TERT KO cells were transfected with the indicated 3Cpro variants and harvested 24 hours post transfection. Conditioned media was analyzed by IL-1β ELISA. **P<0.01 (Two-way ANOVA). n=3 independent transfections. (F) Modes of cell death of Talabostat-treated, or doxycycline-induced Tet-ON HRV14-3Cpro N/TERT cells assayed Annexin VI and PI inclusion. Data are representative of one of two independent experiments.

FIG. 3 shows that 3CPros activate NLRP1 by direct cleavage at a single site between p. Gln130 and Gly131. (A) HRV14-3Cpro cleaves NLRP1 close to its N-terminus. Top panel: the antibodies used to detect the NLRP1 auto-proteolytic fragments. The epitope of the N-terminal NLRP1 antibody is between NLRP1 a.a. 130 and a.a. 230. Bottom panel: 293T cells were transfected with C-terminally HA-tagged NLRP1 and Myc-tagged HRV14-3Pro. Full-length NLRP1 and its cleavage products were visualized with the N-terminal fragment-specific NLRP1 antibody and an antibody against the C-terminal HA tag. Black arrows indicate the proposed proteolytic relationship between the observed NLRP1 fragments. Note that the presence of catalytically active 3Cpro decreased the expression all transfected plasmids (e.g. see also FIG. 2B, 2D). (B) HRV14-3Cpro cleaves NLRP1^{F1212A} at a single site. 293T cells were transfected with FLAG-tagged NLRP1^{F1212A} with increasing amounts of HRV14-3Cpro. Cell lysates were harvested 48 hours post transfection. (C) Recombinant HRV14-3Cpro cleaves human NLRP1. Cell-free lysate (20 μg) from NLRP1-HA-transfected 293T cells were incubated with recombinant HRV14-3Cpro (0.1, 0.3, 1 μg) at 33° C. for 90 mins and analyzed by SDS-PAGE. (D) Mapping of the 3Cpro cleavage site. Top: NLRP1 linker region immediately after the PYRIN domain (PYD). Glutamine (Q) residues are underlined. Bottom: 293T cells were co-transfected with the indicated NLRP1 Q>A mutants and HRV14-3Cpro. 3Cpro^{C146A} was used as a negative control. Total cell lysates were harvested 48 hours post transfection. (E) Q130A abrogates HRV14-3Cpro cleavage. 293T-ASC-GFP-NLRP1^{WT}-FLAG and 293T-ASC-GFP-NLRP1^{Q130A}-FLAG cells were transfected with Myc-HRV14-3Cpro or treated with Talabostat for 48 hours. Total cell lysates were analyzed by SDS-PAGE. *, likely nonspecific NLRP1 degradation product. Black arrow, 3Cpro-dependent cleavage product (a.a. 131-1213). (F) Q130A abrogates 3CPro-dependent, but not Talabostat-dependent NLRP1 activation in 293T cells. Cells were transfected or treated with Talabostat as in FIG. 1E, and fixed 24 hours post transfection. The number of cells with ASC-GFP specks were visually scored with wide-field epifluorescence microscopy at 20× magnification. *P<0.01 ****P<0.0001 (Two-way ANOVA). n=3 independent transfections/drug treatment. (G) Wild-type, but not NLRP1^{Q130A} restores 3Cpro-dependent IL-1β secretion in NLRP1 knockout human keratinocytes. NLRP1 KO keratinocytes were first rescued with stable lentiviral expression of NLRP1^{WT}-FLAG or NLRP1^{Q130A}-FLAG, and further transduced with Tet-ON HRV14-3Cpro. NLRP1 KO or rescued cells were treated with doxycycline (1 μg/mL) or Talabostat (2 μM). Conditioned media were harvested 24 hours after drug treatment and analyzed by IL-1B ELISA. ****P<0.0001 (Two-way ANOVA). n=3 cell seedings and drug treatment. (H) and (I). Wild-type, but not NLRP1^{Q130A} restores 3Cpro-dependent in pyroptosis NLRP1 knockout human keratinocytes. Cells harvested from FIG. 1G were analyzed by Western blots (H) and trypan blue exclusion (I).

FIG. 4 shows that HRV14-3CPro cannot activate murine Nlrp1b. (A) 293T-ASC-GFP were co-transfected with NLRP1^{WT}-HA or NLRP1^{Q130A}-HA together with the indicated proteases. Cell lysates were harvested 48 hours post transfection. (B) Cells treated in S2A were fixed and imaged for ASC-GFP speck formation by wide-field GFP fluorescence. *P<0.05 **P<0.01 (Two-way ANOVA). n=3 independent transfections. (C) Representative morphologies of N/TERT cells expressing HRV14-3Cpro or treated with Talabostat. Inset: cells with ‘membrane ballooning’, a typical feature of lytic cell death. (D) Percentage of Trypan blue positive cells in Tet-ON-HRV14-3Cpro RAW264.7 cells after doxycycline induction of Talabostat (20 μM). (E) Percentage of Trypan blue positive cells after anthrax LF treatment.

FIG. 5 shows that 3Cpro-triggered human NLRP1 activation requires the cullin^{ZER1/ZYG11B} mediated N-terminal glycine degron pathway. (A) Overexpressed NLRP1 (a.a. 131-1474) does not cause spontaneous ASC-GFP speck formation. 293T-ASC-GFP cells were transfected with wild-type NLRP1 or mutants and fixed 48 hours post transfection. (B) Summary of the distinct pathways regulating post-cleavage N-terminal fragment degradation in human NLRP1 and murine Nlrp1b. The two types of the N-degron pathway require distinct recognition receptors, and demonstrate distinct sensitivities to small molecule inhibitors. (C) MLN4924 (NEDD8/cullin inhibitor) and proteasomal inhibitors abrogate 3Cpro-induced ASC-GFP specks. 293T-ASC-GFP-NLRP1-FLAG cells were transfected with HRV14-3Cpro and treated with the indicated drugs for 24 hours. MLN4924, 1 μM. MG132, 2.5 μM and Bortezomib, 0.5 μM, phenylalanine, 1 mM. (D) MLN4924 inhibits NLRP1 self-oligomerization. 293T-NLRP1-FLAG cells were transfected with HRV14-3Cpro and treated with the indicated drugs 16 hours post-transfection for another 24 hours. Whole cell lysate was analyzed by BN-PAGE or SDS-PAGE followed by Western blot. Arrow on right side indicates the HRV14-3Cpro cleavage product (a.a. 131-1213). (E) MLN4924 inhibits endogenous 3Cpro-induced NLRP1 inflammasome and pyroptosis. Tet-ON HRV14-3Cpro N/TERT keratinocytes were treated with doxycycline in combination with the indicated drugs for 24 hours. Cell lysate, conditioned media and 1% NP40 insoluble complexes were analyzed by Western blot. (F) IL-1β ELISA of conditioned media in FIG. 3E. ****P<0.0001. n=3 independent doxycycline inductions/drug treatment. (G) Cullin^{ZER1/ZYG11B}, but not UBR2, is genetically required for 3Cpro-induced NLRP1 activation. The indicated CRISPR KO 293T-ASC-GFP cells were transfected wild-type NLRP1 with HRV14-3Cpro or NLRP1^{M77T}. Cells were fixed 24 hours post-transfection. ASC-GFP specks were scored using wide-field microscopy at 20×. ****P<0.0001, n=3 transfections, >100 cells. (H) Cullin^{ZER1/ZYG11B}, but not UBR2 is responsible for the degradation of the 3Cpro cleavage product. CRISPR KO 293T-ASC-GFP cells stably expressing NLRP1-FLAG were transfected with HRV14-3Cpro. Cell lysate was harvested 48 hours post transfection.

FIG. 6 shows that gain-of-function MLRP1 mutation M77T can destabilize the entire N-terminal fragment to disrupt NLRP1 folding. (A) Expression wild-type or NLRP1 mutants in 293T-ASC-GFP cells. Cell were transfected with the indicated constructs and lysed 48 hours post transfection. (B) Tet-ON HRV14-3Cpro N/TERT cells were pre-treated with the indicated inhibitors and induced with doxycycline (1 μg/mL) for 24 hours. Conditioned media was analyzed by IL-1β ELISA. ****P<0.0001 (Two-way ANOVA). n=3 independent treatment. (C) Representative images of ASC-GFP specks in ZZ-dKO 293T-ASC-GFP cell after transfection with wild-type NLRP1 or NLRP1^{M77T}. Note that ZZ-dKO clone 8 had reduced ASC-GFP expression. ASC-GFP negative cells were not scored for speck formation. (D) Cas9-control and UBR2 KO 293T-ASC-GFP-NLRP1-FLAG cells were transfected HRV14-3Cpro and lysed 48 hours post-transfection.

FIG. 7 shows that NLRP1 is required for inflammasome assembly activation during live HRV infection. (A) Expression levels of NLRP1 vs. NLRP3 (RNAseq FKPM) in NHBEs. n=21, independent cultures of pooled NHBEs from healthy donors. (B) Expression of NLRP1 vs. NLRP3 in healthy human nasal biopsy. Puncta: positive staining by RNAscope. Nuclei staining: hematoxylin. (C) Overview of cytokine profiling of by HRV16-infected and Talabostat-treated NHBEs. (D) Cells were inoculated with HRV16 at MOI=5 and cultured at 33° C. for 48 hours. Luminex array was performed on conditioned media after removing cell debris. n=3 independent infections and treatment. Cytokines/chemokines that were induced at least 5 fold (p<0.05, Student's t test, lognormal values) are represented by square boxes. (E) MCC950 did not affect HRV16 triggered IL-1β secretion in NHBEs. NHBEs were pretreated with MCC950 (5 μM) or Rupintrivir (10 nM) before HRV16 inoculation or Talabostat (2 μM) treatment. (F) MCC950 did not affect HRV16 triggered IL-1β secretion in NHBEs. (G) Overview of apical HRV infection 3D human bronchial epithelial cultures. (H) Alcian blue (AB)/periodic acid-Schiff (PAS) staining of HRV16-infected (MOI=3) and Talabostat (2 μM)-treated 3D human bronchial epithelial cultures. (1) IL-18 level in apical or basal media 48 hours post-infection or Talabostat treatment. **P<0.01, ***P<0.001, ****P<0.0001 (Two-way ANOVA). n=3 independent infected/treated cultures. (J) Secretion of cleaved IL-1β and IL-18 and ASC oligomerization in HRV16-infected primary NHBEs. Cells were inoculated with HRV16 (MOI=5) or treated with Talabostat (2 μM) for 48 hours at 33° C.

FIG. 8 shows that HRV16 infection causes 3Cpro-dependent NLRP1 cleavage and activates the reconstituted human NLRP1 inflammasome. (A) HRV16 infection causes NLRP1 cleavage in HeLa-Ohio-NLRP1-HA cells. MOI=1. (B) Q130A abrogates HRV16-induced NLRP1 cleavage and ASC-GFP oligomerization. HeLa-Ohio-ASC-GFP-NLRP1WT or NLRP1^{Q130A} cells were infected with HRV16 at MOI=1 at 33° C. Cell pellets were harvested 48 hours post infection. (C) The effect of HRV16 infection on ASC-GFP speck formation in HeLa-Ohio-ASC-GFP cells expressing wild-type NLRP1 or NLRP1^{Q130A}. ***P<0.001 (Two-way ANOVA). n=3 replicates from one of two independent infections, >100 cells per condition. (D) Representative images of ASC-GFP specks in HeLa-Ohio-ASC-GFP cells expressing wild-type NLRP1 or NLRP1^{Q130A}. Arrows: ASC-GFP specks. Note that in this cell line the ASC-GFP specks appear less compact, even in Talabostat-treated positive control cells. (E) Expression level of selected inflammasome components and antiviral genes in normal human airway epithelial cells extracted published datasets (Nasal #1-GSE107898; Nasal #2-GSE55458; Bronchial #1-GSE107971; Bronchial #2-GSE107897; Alveolar #1: GSE61220). In datasets where multiple treatment conditions were reported, only the basal/mock conditions were selected. RPM values Nasal #2-GSE55458; Bronchial #1-GSE107971 were logged for heatmap generation. Color mapping, shown in greyscale, was done using the ‘Double gradient’ method in Graphpad Prism 8. (F) IL-18 secretion in HRV16-infected primary human nasal epithelial cells 48 hours post infection (MOI=5).

FIG. 9 shows that HRV16 infection causes NLRP1 activation via direct cleavage and requires the cul^{ZER1/ZYG11B}-mediated N-glycine degron pathway. (A) Endogenous NLRP1, ASC and caspase-1 are indispensable for HRV16-induced IL-18 secretion in NHBEs. Cas9-control, NLRP1, ASC and CASP1 KO NHBEs were infected with HRV16 as before. Conditioned media were harvested 48 hours post-infection. (B) NLRP1 is genetically required for HRV16-induced IL-18 cleavage and ASC oligomerization. Cas9 control, NLRP1 and ASC KO NHBEs were infected with HRV16 as before. Cleaved IL-18 are marked with arrows. (C) Mutating the NLRP1 cleavage site abrogates HRV16-triggered IL-18 secretion in NHBEs. NLRP1 KO NHBEs were rescued with lentiviral constructs expressing FLAG-tagged NLRP1^{WT} or NLRP1^{Q130A}, which carried silent mutations at the PAM sites. The rescued cells were infected with HRV16 or treated with Talabostat. Conditioned media was analyzed by IL-18 ELISA. **P<0.01, ***P<0.001, n=3 replicates from one of two independent infections. (D) Q130A abrogates NLRP1 cleavage, IL-18 maturation and ASC oligomerization in NHBEs. NLRP1 KO or NLRP1^{WT}- or NLRP1^{Q130A}-rescued cells were infected with HRV16 or treated with Talabostat. Cells were harvested 48 hours post infection. Arrow: NLRP1 cleavage product in lane 8 HRV16. *: Non-specific NLRP1 degradation product that occurs right below the a.a. 131-1213 cleavage fragment. See also FIG. 3E, 5E, 5D. (E) MLN4924 and Bortezomib blocks HRV16-induced IL-18 secretion. NHBEs were pre-treated with MLN4924 (1 μM) or Bortezomib (1 μM) before HRV16 infection or Talabostat treatment. IL-18 levels were measured with ELISA. ****, p<0.0001 (Two-way ANOVA), n=3 independent infections/treatment. (F) MLN4924 and Bortezomib blocks HRV16-induced IL-1β secretion. (G) MLN4924 does not affect 3Cpro-dependent VP2 maturation in the course of HRV16 infection. Lysates from HRV16-infected NHBEs in FIGS. 9E-F were analyzed by Western blot 48 hours post HRV16 inoculation.

FIG. 10 shows further results that NLRP1 is the primary inflammasome sensor for HRV infection. (A) Caspase-1 activity in the conditioned media of HRV16-infected, or Talabostat-treated NHBEs. ****P<0.0001 (Two-way ANOVA), n=4 replicates of one of 2 independent infections. (B) Caspase-1 activity in the conditioned media of HRV16-infected, or Talabostat-treated NHBEs. ****P<0.0001, **P<0.01 (Two-way ANOVA). n=3 infections. (C) Representative morphology of HRV16 infected or Talabostat-treated NHBEs. Black arrows indicate membrane ballooning typical of lytic cell death. Lower panel intentionally focuses on floating cells; hence the attached monolayer appears out of focus. (D) Western blot validation of ASC and CASP1 KO in NHBEs. (E) Genomic sequencing validation of NLRP1 KO in NHBEs. (F) and (G). LDH release and IL-8 secretion in HRV16-infected or Talabostat-treated KO NHBEs. **P<0.01, ***P<0.001 (Two-way ANOVA). n=3 infections/treatment. ****P<0.0001 (Two-way ANOVA).

FIG. 11 shows that NLRP1 functions in parallel with other immune sensors such as TLRs and RLRs. (A) Proposed model for HRV-triggered NLRP1 activation in the human airway epithelium. (B) Sequence conservation of the 3Cpro cleavage site among primate species. The minimal cleavage site (Q-G) is shaded.

### DETAILED DESCRIPTION OF THE INVENTION

Bibliographic references mentioned in the present specification are for convenience listed in the form of a list of references and added at the end of the examples. The whole content of such bibliographic references is herein incorporated by reference. Any discussion about prior art is not an admission that the prior art is part of the common general knowledge in the field of the invention. The present invention relates to the identification of a PAMP that can trigger the NLRP1-activated inflammasome in human airways. More particularly, the inventors have identified the NLRP1-activated inflammasome pathway, involving cullin^{ZER1/ZYG11B} and NEDD8-activating enzyme (NAE), and potential inhibitors of same.

### Definitions

Certain terms employed in the specification, examples and appended claims are collected here for convenience.

It must be noted that as used herein and in the appended claims, the singular forms “a”, “an”, and “the” include plural reference unless the context clearly dictates otherwise.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The terms “nucleotide”, “nucleic acid” or “nucleic acid sequence”, as used herein, refer to an oligonucleotide, polynucleotide, or any fragment thereof, to DNA or RNA of genomic or synthetic origin which may be single-stranded or double-stranded and may represent the sense or the antisense strand, to peptide nucleotic acid (PNA), or to any DNA-like or RNA-like material.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

Salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

The term “treatment”, as used in the context of the invention refers to prophylactic, ameliorating, therapeutic or curative treatment.

The term “subject” is herein defined as vertebrate, particularly mammal, more particularly human. For purposes of research, the subject may particularly be at least one animal model, e.g., a mouse, rat and the like. For example, for treatment of airway inflammation and related disorders the subject may be a human with an Enterovirus infection.

Bibliographic references mentioned in the present specification are for convenience listed in the form of a list of references and added at the end of the examples. The whole content of such bibliographic references is herein incorporated by reference.

A person skilled in the art will appreciate that the present invention may be practiced without undue experimentation according to the methods given herein. The methods, techniques and chemicals are as described in the references given or from protocols in standard biotechnology and molecular biology text books.

Having now generally described the invention, the same will be more readily understood through reference to the following examples which are provided by way of illustration, and are not intended to be limiting of the present invention.

### EXAMPLES

### Example 1

### Materials and Methods

### Cell Culture

293 Ts (ATCC #CRL-3216), HeLa-Ohio (ECACC General Cell Collection #84121901) and normal bronchial epithelial cells (Lonza #CC-2541) were obtained from commercial sources and cultured according to the suppliers' protocols. Immortalized human keratinocytes (N/TERT-1, or N/TERT herein) were a kind gift from H. Reinwald (MTA) [Mihaylova et al., Cell Rep. 24: 3000-3007.e3 (2018)]. All cell lines underwent routine Mycoplasma testing with Lonza MycoAlert (Lonza #LT07-118).

### Plasmid Transfection and Stable Cell Line Generation Using Lentiviruses

293T-ASC-GFP, N/TERT-ASC-GFP, N/TERT NLRP1 KO cells were described previously [Zell et al., Arch. Virol. 163: 299-317 (2018)]. All transient expression plasmids were cloned into the pCS2+ vector using standard restriction cloning using ClaI and XhoI. Polyclonal Cas9/CRISPR knockout cell lines were generated with lentiCRISPR-v2 (Addgene #52961) and selected with puromycin. Knockout efficiency was tested with Western blot 7-10 days after puromycin selection. Site-directed mutagenesis was carried out with QuickChangeXL II (Agilent #200522). Constitutive lentiviral expression was performed using pCDH vectors (SystemBio). Doxycycline-inducible Tet-ON lentiviral constructs were based on the pTRIPZ backbone (ThermoFisher).

### Antibodies and Cytokine Analysis

The following antibodies were used in this study: c-Myc (Santa Cruz Biotechnology #sc-40), HA tag (Santa Cruz Biotechnology, #sc-805), GAPDH (Santa Cruz Biotechnology, #sc-47724), ASC (Adipogen, #AL-177), CASP1 (Santa Cruz Biotechnology, #sc-622), IL1B (R&D systems, #AF-201), FLAG (SigmaAldrich, #F3165), GFP (Abcam, #ab290), NLRP1 (R&D systems, #AF6788), IL18 (Abcam ab207324) and VP2 (QED Bioscience, #18758). HRV16-3Cpro was detected by rabbit serum, a kind gift from Dr. Ann Palmenberg and Dr. James Gern (University of Wisconsin). Cytokine and chemokine measurements were carried out with human IL-1B ELISA kit (BD, #557953), human IL-18 ELISA kit (MBL Bioscience, #7620) and Immune Monitoring 65-Plex Human ProcartaPlex™ Panel (ThermoFisher EPX650-10065-901).

### HRV16 Virus Propagation

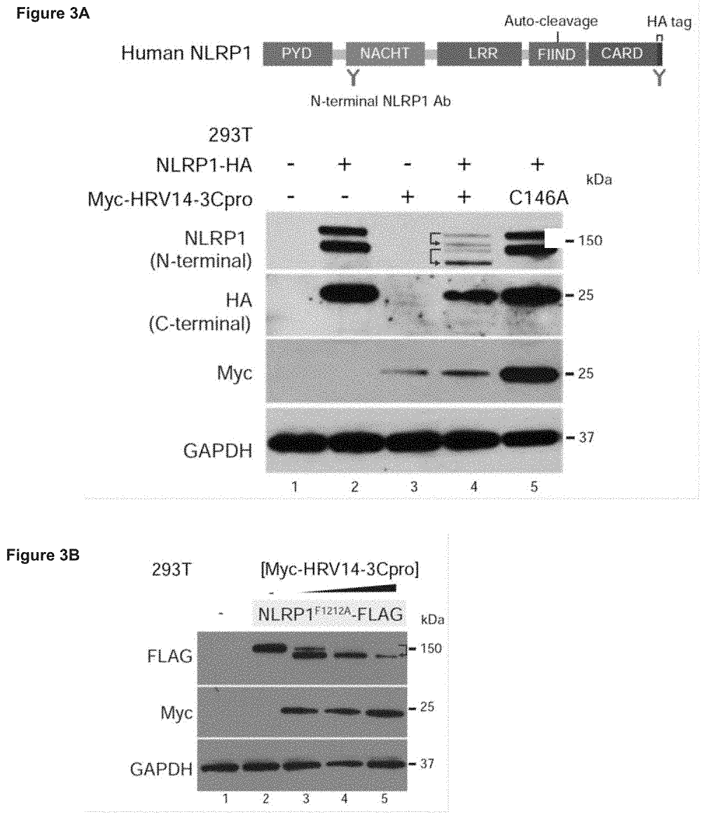

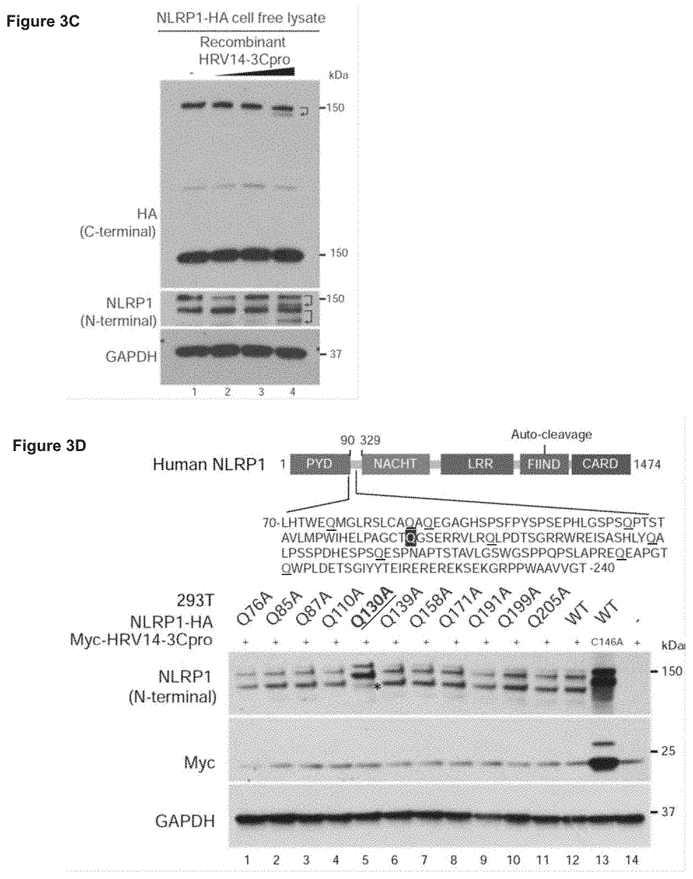

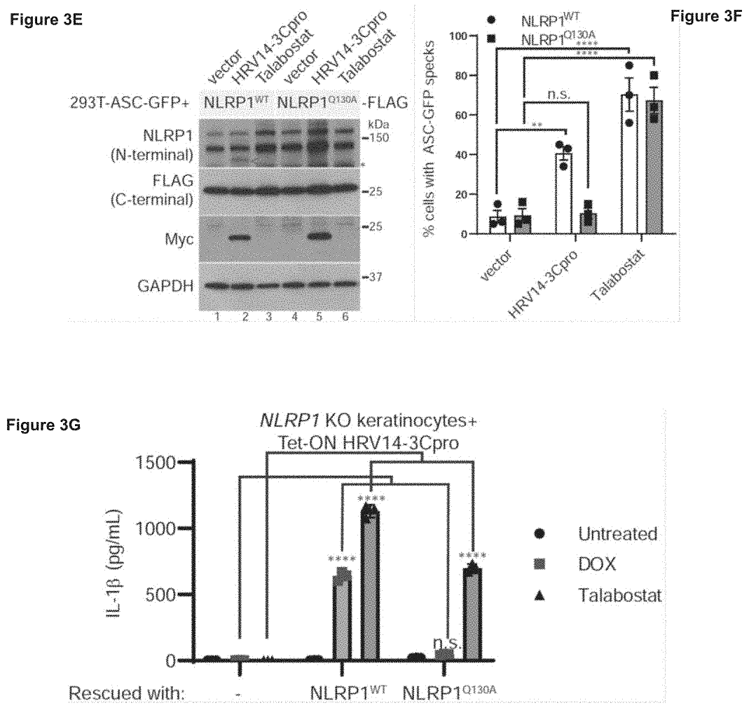

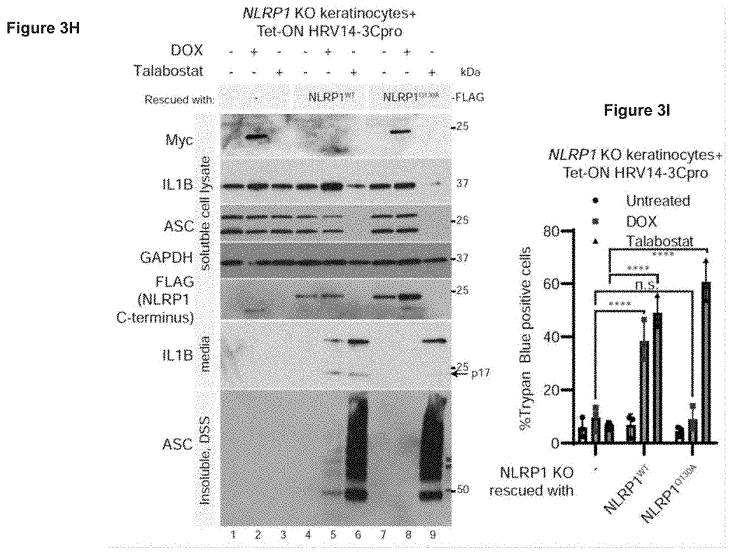

HRV used in the study was HRV-A16 (strain 11757; ATCC VR-283, Manassas, Va., USA), and was propagated in HeLa cell line (HeLa Ohio, ECACC 84121901, Porton Down, Salisbury, Wiltshire, UK). HeLa cells were grown in Eagle's Minimum Essential Medium (EMEM) ATCC® 30-2003™, supplemented with 10% fetal bovine serum (FBS) (BioWest, Kansas City, Mo., USA), 2% HEPES and 1% Antibiotic-Antimycotic (Anti-Anti) (Gibco) and incubated at 37° C. humidified incubator with 5% CO₂. To propagate HRV16, HeLa cells were first seeded to achieve confluency of about 80-90% in 24-well plate overnight (FIG. 3A). Cells were rinsed with 1× dulbecco's phosphate-buffered saline (dPBS) and infected with HRV16 before addition of EMEM with 2% FBS, 2% HEPES and 1% Anti-Anti. Infected HeLa cells were incubated at 33° C. for two to three days. Viruses were harvested from the supernatants of infected HeLa cells when about 80% cytopathic effects (CPE) were observed (FIG. 3B). HRV virus stocks were centrifuged at 3500 rpm for 10 mins at 4° C. to remove cellular debris, and aliquoted into cryovials for storage at −80° C.

### Inoculation of Human Rhinovirus

HRV was diluted using the respective cell culture medium and inoculated at multiplicity of infection (MOI) of 5.0 (NHBE) and 1.0 (HeLa), respectively. Infected cells were incubated at 33° C. for 1 hr. Conditioned non-infected cell culture medium from viral propagation was added as uninfected-control. The HRV-infected and control cells were then incubated at 33° C. for up to 48 hours post-infection (hpi). Cell culture supernatant and cell lysate were collected to perform relevant assays between 24-72 hpi.

### Viral Quantification Using Rhinovirus Plaque Assay

HeLa cells (at 85-95% confluence) in 24-well plates were incubated with 100 μL of serial dilutions from 10⁻¹ to 10⁻⁶ of sample from infected hNECs at 33° C. for 1 h. The plates were rocked every 15 min to ensure equal distribution of virus. The inoculum was removed and replaced with 1 mL of Avicel (FMC Biopolymer) overlay to each well, and incubated at 33° C. for 65-72 h. The overlay components were optimized to obtain HRV plaques suitable for counting. Avicel powder was added into double strength MEM to formulate 1.2% Avicel solution, and with a final concentration comprising 3% FBS, 2% HEPES, 1.5% NaHCO₃, 3% MgCl₂ and 1% Anti-Anti. Avicel overlay was removed after the incubation period, and cells were fixed with 20% formalin in 1×PBS for 1 h. Formalin was removed, and cells were washed with 1×PBS. The fixed cells were stained with 1% crystal violet for 15 min, and washed. The plaque-forming units (PFU) were calculated as follows: Number of plaques×dilution factor=number of PFU per 100 μL, which is then expressed as PFU/mL.

### HRV16 Infection of 3D Reconstructed Human Bronchial Epithelium

3D culture of bronchial epithelium was purchased from Mattek (AIR-1484 AIR-100 EpiAirway, 3D Respiratory Epithelial Human MicroTissues) and cultured using the Extended Culture protocol as advised by the supplier.

### CRISPR/Cas9 Knockout

CRISPR/Cas9 editing was performed in 293T cells was performed according to the method reported by the Doyon group [Agudelo et al., Nat Methods. 14: 615-620 (2017)], incorporated herein by reference, except that guide RNAs (sgRNAs) were cloned into pSpCas9(BB)-2A-Puro (PX459) V2.0 (Addgene 62988). N/TERT and NHBE KOs were performed using LentiCRISPR-V2 (Addgene 52961) and stable lentiviral transduction. The sgRNAs used are shown in Table 1.

TABLE 1
List of sgRNA

| sgRNA used | | SEQ |
|---|---|---|
| (Gene #sgRNA number) | Sequence | ID NO. |
| NLRP1#1 | GATAGCCCGAGTGACATCGG | 1 |
| NLRP1#2 | AGCCCGAGTGACATCGGTGG | 2 |
| CASP1#1 | ACAGACAAGGGTGCTGAACA | 3 |
| CASP1#2 | ATTGACTCCGTTATTCCGAA | 4 |
| CASP1#3 | TGACTCCGTTATTCCGAAAG | 5 |
| ASC#2 | GCTAACGTGCTGCGCGACAT | 6 |
| ASC#3 | CATGTCGCGCAGCACGTTAG | 7 |
| UBR2#1 | TGCATAACTTGAACTTTGAG | 8 |
| UBR2#2 | TCCATGCACAAAACACAAGT | 9 |
| UBR2#3 | ACTGTGGTGATACTGAAGCC | 10 |
| UBR2#4 | TGATACTGAAGCCTGGAAAG | 11 |
| UBR2#5 | TTGCATGCTGTTTAATGATG | 12 |
| ZER1#5 | CAGGGACCCAATCAATCATG | 13 |
| ZER1#6 | GGCAGGACGAGTCTATCCAG | 14 |
| ZER1#7 | AGGCTGAAGAAGCTCTCGTG | 15 |
| ZYG11B#5 | AGCGCTCGTAAGGATCCTCG | 16 |
| ZYG11B#6 | GCAGTGGCTTTGCAACCATG | 17 |
| ZYG11B#7 | CTTGGTTAAGTTAAATACAC | 18 |

### Example 2

### Enteroviral 3Cpros Activate the Human NLRP1 Inflammasome

In a survey of common human pathogens, we considered the human rhinovirus (HRV), the causative agent for the common cold. HRV is a member of the enterovirus genus (family: Picornaviridae), a family of single-stranded RNA viruses that cause a wide range of human diseases, including hand-foot-and-mouth disease, peri/myocarditis and poliomyelitis [Zell, Arch. Virol. 163: 299-317 (2018)]. Importantly, HRV infection of primary human bronchial epithelial cells has been reported to induce caspase-1 activation and IL-1 secretion [Zell, Arch. Virol. 163: 299-317 (2018); Piper et al., PLoS One 8: e63365 (2013)], although the upstream sensing mechanisms were not clear. Notably, all picornaviruses, including HRVs, encode two well-defined proteases termed 2Apro and 3Cpro, which are required to cleave the viral genomic precursor proteins into individual functional components [Palmenberg, Annu. Rev. Microbiol. 44: 603-623 (1990)]. Both proteases have also been reported to cleave host proteins to facilitate viral replication or immune evasion [Walker et al., PLoS One 8: e71316 (2013); Croft et al., Sci. Rep. 8: 1569 (2018); de Breyne et al., Virology 378: 118-122 (2008); Mukherjee et al., PLoS Pathogens 7: e1001311 (2011)].

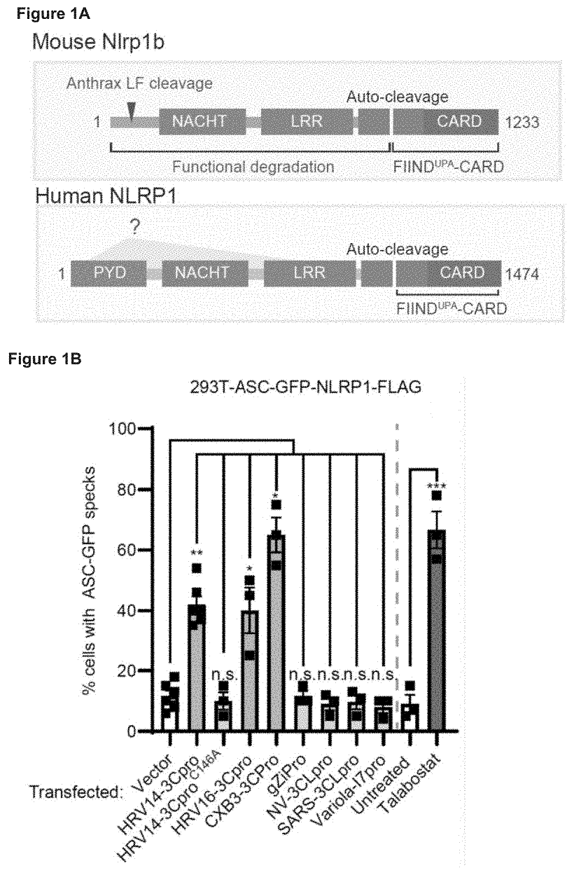

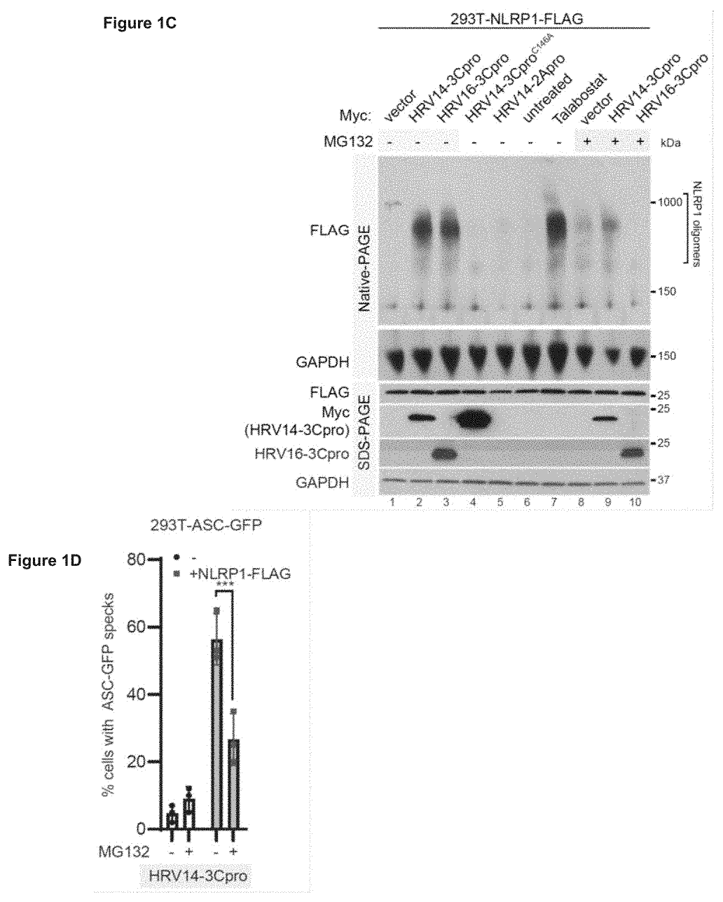

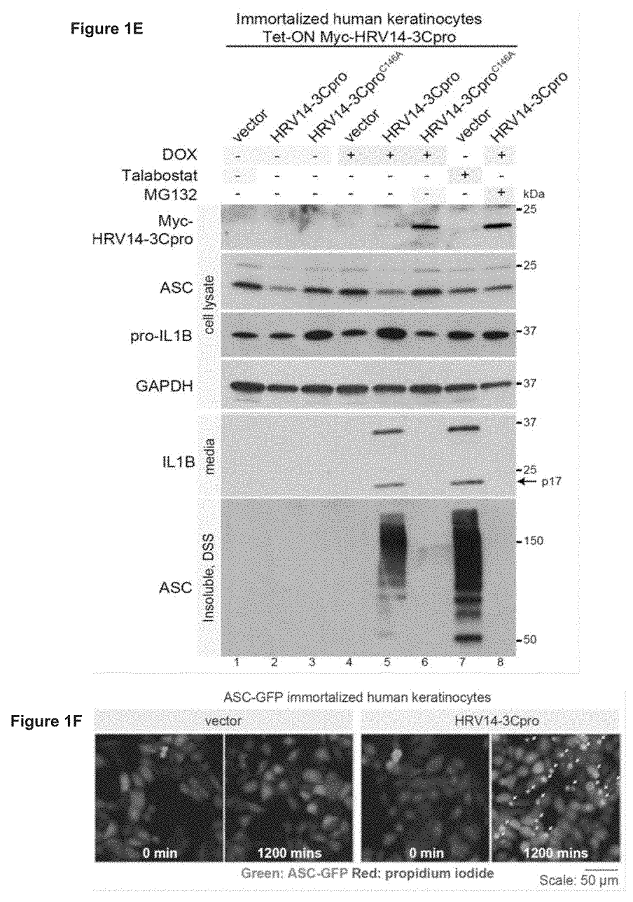

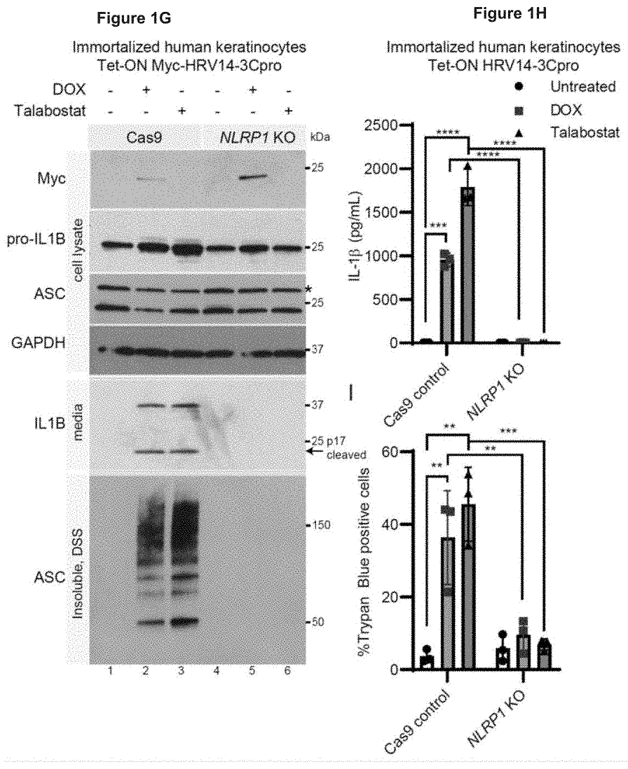

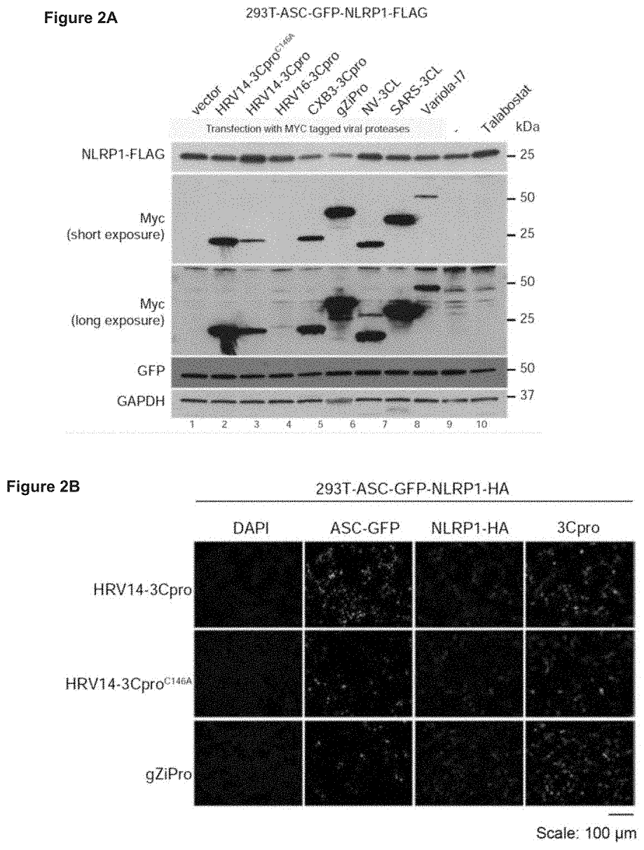

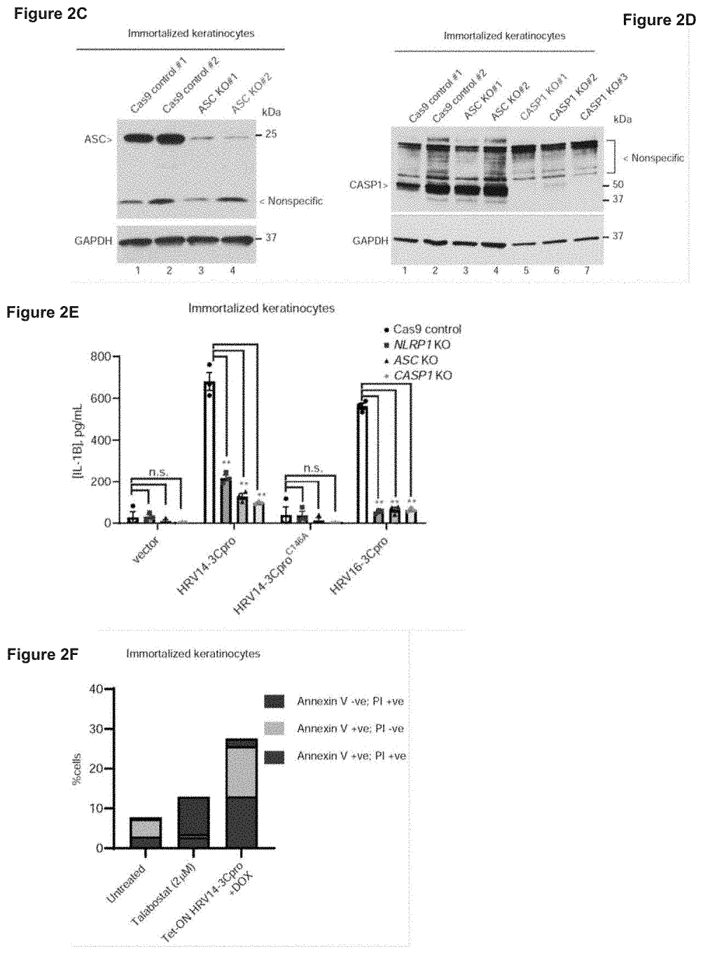

To test whether HRV-3Cpro can activate human NLRP1, we expressed Myc-tagged HRV-3Cpro in a 293T reporter cell line that stably expressed ASC-GFP and NLRP1-FLAG (termed 293T-ASC-GFP-NLRP1-FLAG). As compared to vector-transfected cells, 3Cpros derived from two strains of HRV (HRV-14, serotype B and HRV16, serotype A) and a closely related enterovirus (coxsackie B3) caused a significant increase in the percentage of cells forming ASC-GFP specks (FIG. 1B, FIG. 2A-B), similar to the DPP8/9 inhibitor, Talabostat. In contrast, none of the other viral proteases tested, including Zika NS2B-3 (gZiPro), norovirus (NV) 3CLpro, SARS-3CLpro and smallpox (Variola) 17pro, was able to do so (FIG. 1B, FIG. 2A-B), despite similar or even higher levels of expression. The ability of HRV14-3Cpro to activate human NLRP1 was entirely dependent on its enzymatic activity, since mutating its catalytic cysteine residue (p.C146A) abrogated ASC-GFP speck formation (FIG. 1B, FIG. 2B). In an orthogonal assay for NLRP1 activation, HRV14- and HRV16-3Cpro directly induced the formation of high-molecular weight NLRP1 oligomers at the expense of monomeric NLRP1 (FIG. 1C, lanes 2, 3 vs. lane 1) by Native-PAGE. We could not reliably assay the effect of HRV-2Apro on human NLRP1, as Myc-tagged 2Apro did not accumulate in 293T cells (FIG. 1C, lane 5). These findings establish enteroviral 3Cpros, such as those encoded by HRV, as robust viral activators for reconstituted human NLRP1 inflammasome in vitro. Furthermore, the effect of 3Cpro requires continuous proteasome activity, as the proteasomal inhibitor MG132 significantly blocked HRV-3Cpro-induced ASC-GFP speck formation (FIG. 1D) in 293T-ASC-GFP-NLRP1 cells and NLRP1 self-oligomerization (FIG. 1C, lanes 1-3 vs. 8-10). Hence, despite very different pathogen origins and substrate specificities, 3Cpro and anthrax LT likely trigger human NLRP1 and rodent Nlrp1b, respectively, via a common mechanism that involves cleavage followed by ‘functional degradation’.

Recently it has been demonstrated that primary and immortalized human keratinocytes express components of the NLRP1 inflammasome complex endogenously and therefore undergo rapid pyroptosis upon Talabostat treatment [Zhong et al., Cell 167: 187-202.e17 (2016); Zhong et al., J. Biol. Chem. 293: 18864-18878 (2018)]. This provides a robust cellular system to determine if 3Cpros can activate the endogenous NLRP1 inflammasome. Immortalized human keratinocytes were stably transduced with doxycycline-inducible (Tet-ON) lentiviral constructs encoding dsRed (vector control), HRV14-3Cpro or its catalytically inactive mutant C146A (FIG. 1E). Upon doxycycline treatment, only cells expressing active HRV14-3Cpro demonstrated cardinal features of pyroptosis, including the secretion of cleaved, mature IL-1β and the formation of detergent-insoluble ASC oligomers (FIG. 1E, lanes 5 vs. lanes 1-4 and lane 6). This occurred in spite of lower expression of wild-type 3Cpro than its inactive mutant (p.C146A) (FIG. 1E, lane 5 vs. 6). Pre-treatment with MG132 completely abrogated IL-1β secretion and ASC oligomerization (FIG. 1E, lane 5 vs. lane 8). Live-cell imaging of human keratinocytes expressing GFP-tagged ASC further corroborated that HRV14-3Cpro could induce ASC-GFP speck formation (FIG. 1F). HRV14-3Cpro-induced pyroptosis was entirely dependent on NLRP1, as its genetic ablation by CRISPR/Cas9 (NLRP1 KO Tet-ON HRV14-3Cpro) abrogated IL-1 secretion, ASC oligomerization (FIG. 1G, lane 2-3 vs. lane 5-6, FIG. 1H) and lytic cell death (FIG. 1I) following doxycycline induction. In experiments with transiently transfected HRV14- and HRV16-3Cpro, NLRP1, ASC and CASP1 deletion all had similar inhibitory effects (FIG. 2C-E). Therefore, HRV-3Cpro can act as a potent trigger of pyroptotic cell death in immortalized human keratinocytes by activating the endogenous NLRP1 inflammasome. Similar to reconstituted NLRP1 in 293T cells (FIG. 1C-D), this effect also required intact proteasome activity (FIG. 1E, lane 7 vs. lane 8), suggesting that a ‘functional degradation’ mechanism is likely at play. We also observed that a subset of 3Cpro-expressing keratinocytes underwent apoptotic cell death with Annexin V staining but without PI inclusion (FIG. 2F). This observation is in agreement with the reported pro-apoptotic roles of 3Cpros [Buenz and Howe, Trends in Microbiology 14: 28-36 (2006); Croft et al., mBio 8 (2017)] in other cell types. By contrast, Talabostat exclusively caused lytic cell death (FIG. 2F) marked by PI inclusion.

### Example 3

### 3Cpros Activate NLRp1 by Direct Cleavage at a Single Site Between Gln130 and Gly131

Picornaviral 3Cpros, including HRV14-3Cpro, are cysteine proteases with well-defined catalytic activity and broad substrate preferences [Palmberg, Annu. Rev. Microbiol. 44: 603-623 (1990); Matthews et al., Cell 77: 761-771 (1994)]. Just as anthrax LF cleaves rodent Nlrp1b directly (Chavarria-Smith and Vance, PLoS Pathog. 9: e1003452 (2013); Chavarria-Smith et al., PLoS Pathog. 12: e1006052 (2016)), we hypothesized that 3Cpros could activate human NLRP1 via direct cleavage. Overexpressed NLRP1 undergoes auto-cleavage within its FIND and thus appears as two bands that differ by ˜20 kDa when visualized with an N-terminal specific antibody [Finger et al., J Biol. Chem. 287: 25030-25037 (2012); D'Osualdo et al., PLoS One 6: e27396 (2011)] (FIG. 3A, lane 2). In the presence of HRV14-3Cpro, two additional bands became apparent using the same antibody (FIG. 3A, lane 4), while the C-terminal FIIND^{UPA}-CARD fragment remained intact, suggesting that the HRV14-3Cpro cleaved NLRP1 at a single site close to the N-terminus, and not within the FIIND^{UPA}-CARD fragment. To visualize the 3Cpro-specific cleavage more precisely, the same experiment was carried out using the NLRP1^{F1212A} mutant, which cannot undergo auto-cleavage within the FIIND and thus remains as a single band by SDS-PAGE (FIG. 3B, lane 2). With increasing amounts of HRV14-3Cpro, NLRP1^{F1212A} became cleaved into a single proteolytic product, which was approximately 20 kDa smaller than full-length NLRP1 (FIG. 3B). In both experiments, the proteolytic banding patterns could be explained by a single cleavage site approximately ˜20 kDa from the NLRP1 N-terminus. The same cleavage could be observed when NLRP1-expressing cell-free lysate was incubated with recombinant HRV14-3Cpro at 33° C. (the preferred temperature for HRV infection), suggesting that the observed cleavage was most likely direct (FIG. 3C).

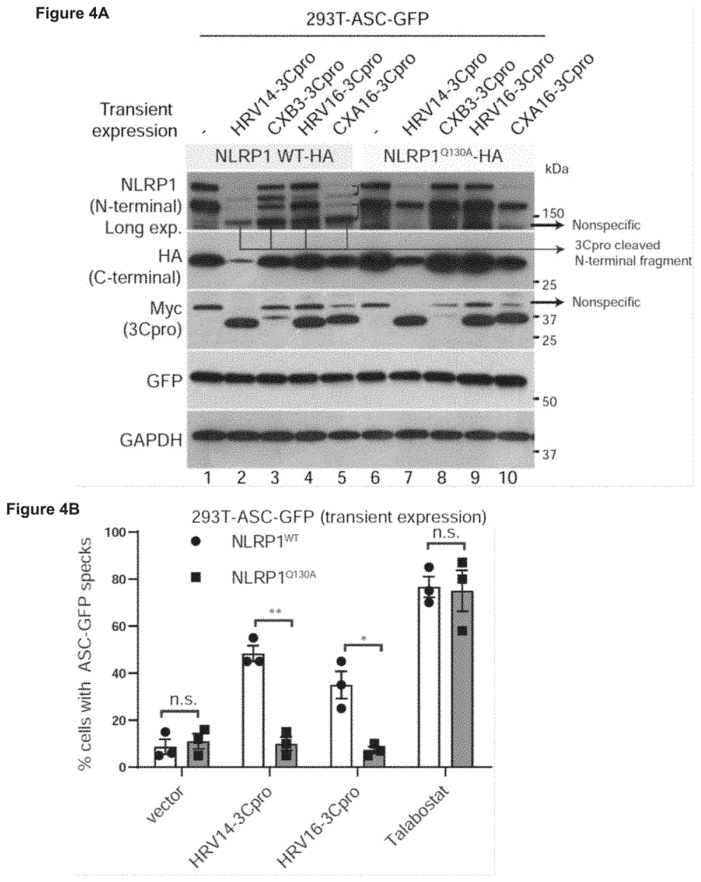

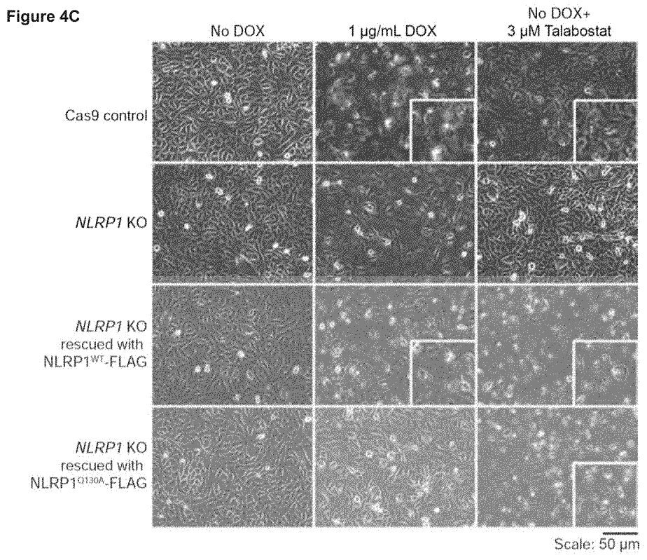

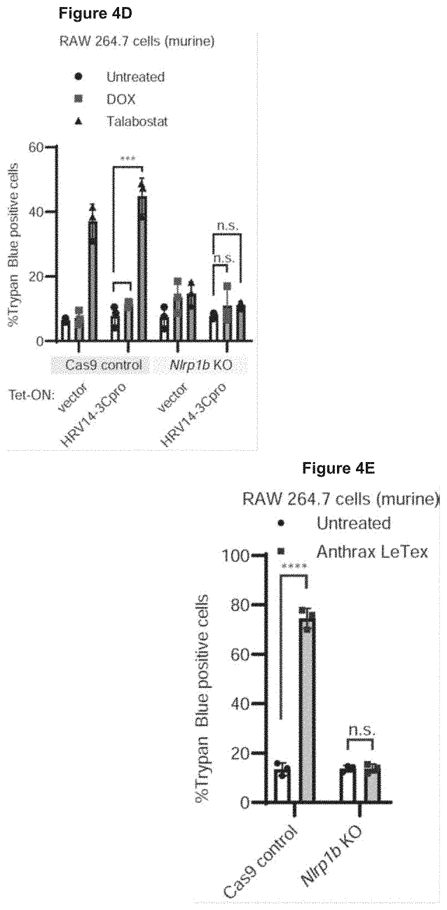

Based on the size difference between cleaved and full-length NLRP1, the 3Cpro cleavage site was mapped to the linker region immediately after the PYRIN domain (PYD) (FIG. 3D, top panel), a region that is not conserved in rodents. As 3Cpros require a glutamine residue at the P′-1 substrate site [Matthews et al., Cell 77: 761-771 (1994)], each of the 11 glutamine residues present in this linker region was changed to alanine by site-directed mutagenesis. Of all the alanine mutants tested, only the Q130A missense mutation abrogated NLRP1 cleavage by HRV14-3Cpro (FIG. 3D, lane 5 vs. other lanes) and other 3CPros (FIG. 4A, lanes 1-5 vs lanes 6-10). Next we generated reporter 293T-ASC-GFP cells that stably expressed either wild-type NLRP1 or the uncleavable Q130A mutant (NLRP1^{Q130A}) (FIG. 3E, lane 2 vs. lane 5). The 3Cpro-dependent cleavage band was only observed in wild-type NLRP1 expressing cells (FIG. 3E, lane 2, black arrow) and therefore likely derived from a.a. 131-1213. In contrast to wild-type NLRP1, NLRP1^{Q130A}-expressing cells no longer nucleated ASC-GFP specks in response to HRV14-3Cpro expression; however, its response to Talabostat remained intact relative to wild-type NLRP1 (FIG. 3F). Similar results were obtained using transfected NLRP1^{Q130A} and HRV16-3Cpro (FIG. 4B).

To study the endogenous inflammasome response, we rescued NLRP1 KO human keratinocytes with either wild-type NLRP1 or the cleavage site mutant, NLRP1^{Q130A}. The ‘rescued’ cells were further transduced with Tet-ON HRV14-3Cpro lentiviruses. Only wild-type NLRP1, but not NLRP1^{Q130A}, restored HRV14-3Cpro-triggered IL-1β secretion, ASC oligomerization (FIG. 3G, 3H, lane 5 vs. lane 8) and lytic cell death (FIG. 31, FIG. 4C), while both wild-type NLRP1 and NLRP1^{Q130A} re-enabled NLRP1 KO cells to respond to Talabostat (FIG. 3G, FIG. 3H, lane 6 vs. lane 9, FIG. 31, FIG. 4C). These results establish that 3Cpro-triggered NLRP1 activation is a direct result of a discrete cleavage site between a.a. Q130 and a.a. G131 adjacent to the human-specific PYRIN domain. Since this site is not conserved in rodents, HRV-3Cpro would not be expected to activate murine Nlrp1b. This is consistent with the fact that HRV, similar to most pathogenic human enteroviruses, do not naturally infect rodents. To test this experimentally, Tet-ON HRV14-3Cpro-expressing wild-type and Nlrp1b KO murine RAW264.7 cells were generated. Doxycycline induction failed to induce pyroptotic cell death in either wild-type or Nlrp1b KO RAW264.7 cells. These results confirm that HRV14-3Cpro cannot activate murine Nlrp1b (FIG. 4D-E).

### Example 4

### 3Cpro-Triggered Human NLRP1 Activation Requires the Cullin^{ZER1/ZYG11B} Mediated N-Terminal Glycine Degron Pathway

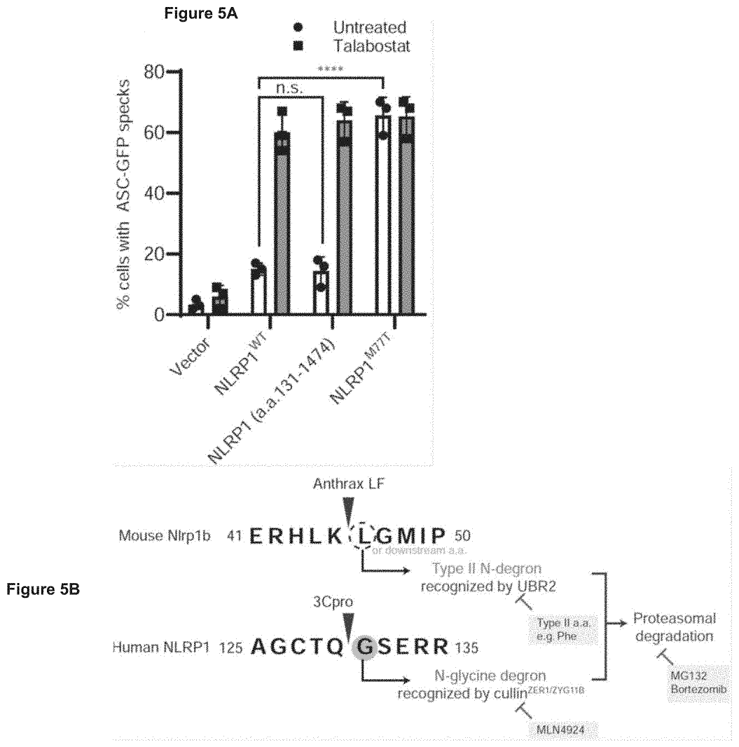

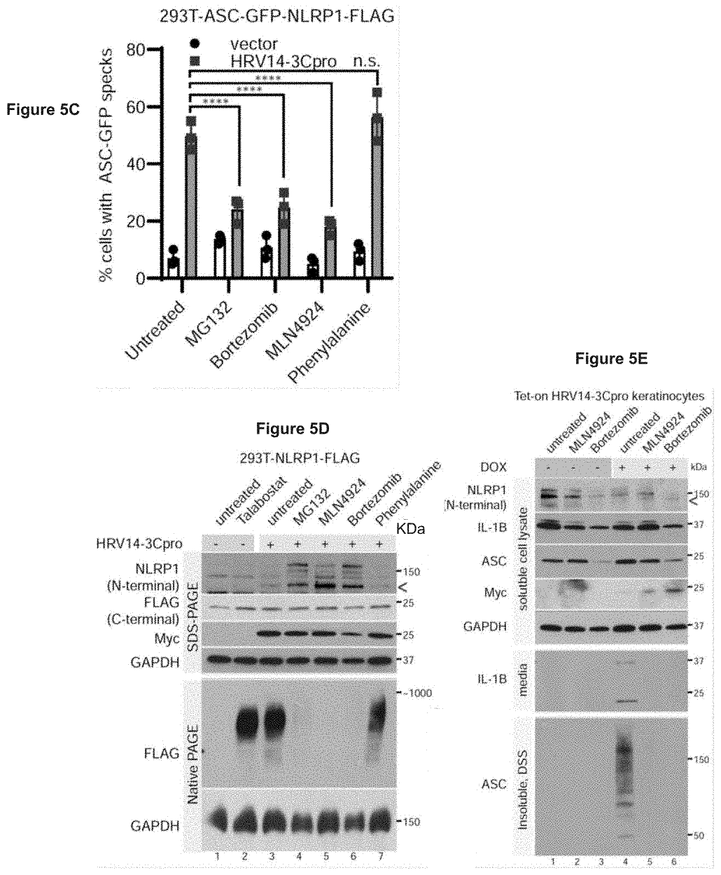

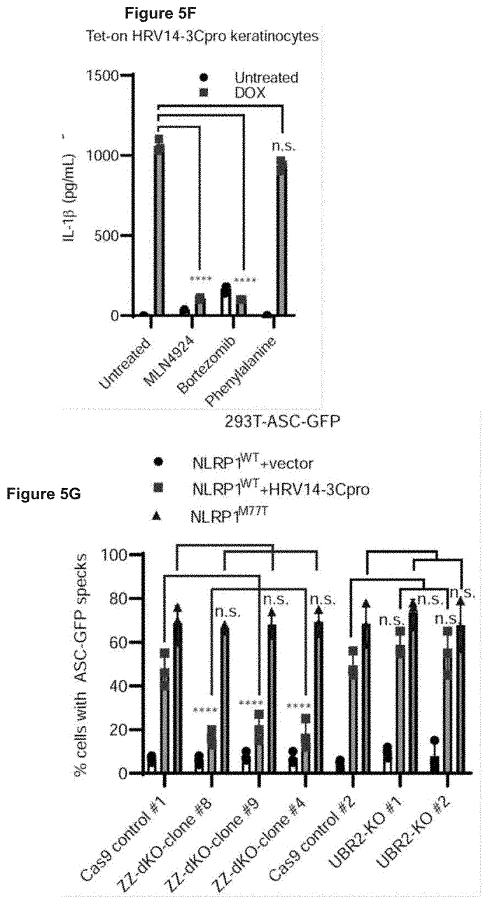

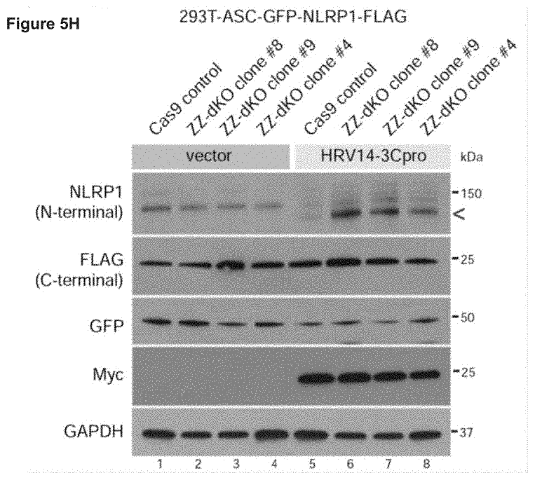

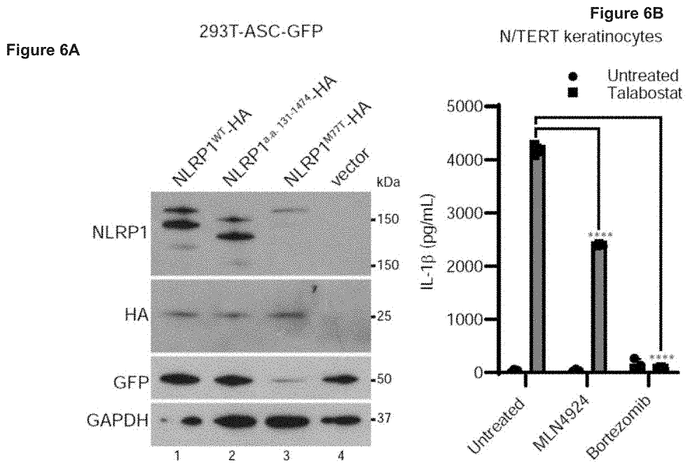

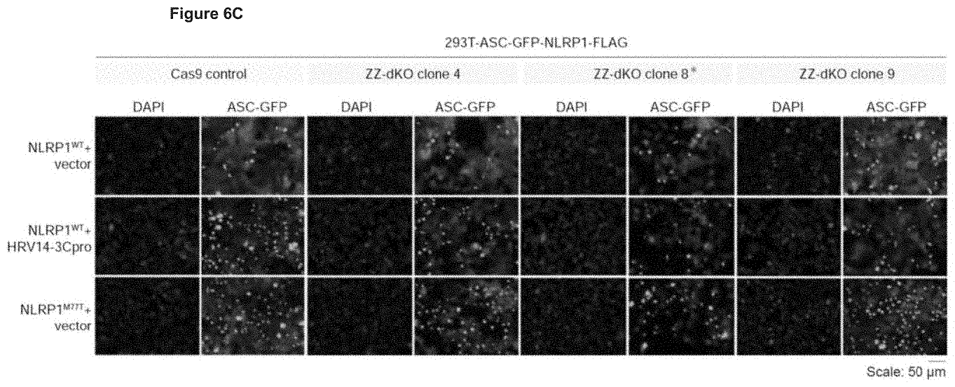

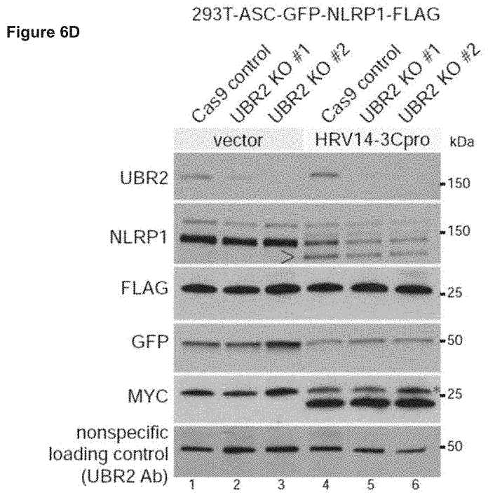

We sought to further dissect how the cleavage of human NLRP1 by 3Cpros triggers proteasome-dependent inflammasome activation. In contrast to anthrax LF-mediated cleavage of murine Nlrp1b, HRV-3Cpro removes the entire human-specific NLRP1 PYRIN domain (PYD) where most disease-causing, gain-of-function germline mutations are located (Grandmange et al. Annals of the Rheumatic Diseases 76: 1191-1198 (2017); Zhong et al., Cell 167: 187-202.e17 (2016); Drutman et al., PNAS USA 116: 19055-19063 (2019); Herlin et al., Rheumatology (2019), doi:10.1093/rheumatology/kez612). Several possible mechanisms could account for how 3Cpro cleavage activates NLRP1. For instance, it could relieve the auto-inhibitory effect that is intrinsic to the PYD domain, or 2) the cleavage could trigger the destabilization of the largest fragment after cleavage (between the 3Cpro cleavage site and the FIND auto-proteolysis site, a.a. 131-1213). To distinguish these two possibilities, we examined a truncation mutant of NLRP1 (a.a. 131-1474), which mimics the major product generated by HRV14-3Cpro cleavage except for the initiating methionine. Unexpectedly, this mutant did not cause increased ASC-GFP specks formation relative to wild-type NLRP1 when expressed in 293T-ASC-GFP reporter cells. Furthermore, it remained fully sensitive to Talabostat-mediated activation (FIG. 5A, FIG. 6A, lane 1 vs. lane 2). These results argue against the first scenario and suggest that the removal of NLRP1 PYD is not sufficient in itself to account for HRV-3Cpro triggered NLRP1 activation.

Recently a number of reports have characterized the mechanism by which anthrax LF cleavage triggers Nlrp1b activation [Chui et al., Science 364: 82-85 (2019); Sandstrom et al., Science 364 (2019), doi:10.1126/sciencedotaau1330; Xu et al., EMBO J. 38: e101996 (2019)]. LF cleavage creates an N-terminal degron that is recognized by the Type II N-degron receptors, such as UBR2, which degrade the inhibitory Nlrp1b N-terminal fragment via the proteasome (FIG. 5B). These findings resolved a long-standing question in the field as to why Nlrp1b activation could be blocked by certain free amino acids, such as phenylalanine and leucine [Wickliffe et al., Cell Microbiol. 10: 1352-1362 (2008)], which are competitive inhibitors of UBR proteins. Inspired by these findings, we hypothesized that an analogous pathway might account for the ability of HRV-3Cpro to activate human NLRP1. However, HRV-3Cpro cleavage of human NLRP1 generates a fragment (a.a. 131-1273) bearing an N-terminal glycine, which is not a canonical type II N-terminal degron recognized by related UBR proteins [Wickliffe et al., Cell Microbiol. 10: 1352-1362 (2008); Varshavsky, PNAS USA 116: 358-366 (2019)]. While this manuscript was under review, a glycine-specific N-degron pathway was described [Timms et al., Science 365 (2019)]. N-terminal glycine residues are recognized by receptors ZER1 and ZYG11B and their partner cullins, CUL2 and CUL5 (termed cullin^{ZER1/ZYG11B}). The cullin^{ZER1/ZYG11B} machinery ubiquitinates substrate proteins with N-terminal glycine residues and causes their degradation via the proteasome. In contrast to the UBR system, the N-glycine degron pathway is not sensitive to type II free amino acids, but can be inhibited by the NEDD8/cullin inhibitor MLN4924 [Timms et al., Science 365 (2019)](FIG. 5B). Using a series of chemical inhibitors, we tested whether the newly described N-glycine degron pathway contributes to HRV-3Cpro triggered human NLRP1 activation. In 293T-ASC-GFP-NLRP1 reporter cells, MLN4924 blocked HRV14-3Cpro-induced ASC-GFP speck formation (FIG. 5C), similar to proteasomal inhibitors MG132 and Bortezomib. MLN4924 similarly blocked HRV14-3Cpro NLRP1 self-oligomerization (FIG. 5D, lane 5 vs lane 3) in 293T-NLRP1-FLAG cells. Most notably, it led to the stabilization of the post-cleavage NLRP1 fragment (corresponding to a.a. 131-1213 taking into account FIIND auto-proteolysis), to similar degrees as did MG132 and Bortezomib (FIG. 5D, lane 4-6 vs. lane 3, black arrow). Type II N-degron inhibitor phenylalanine had no effect in either ASC-GFP speck formation or NLRP1 self-oligomerization (FIG. 5C, FIG. 5D, lane 7). Similar effects were observed for the endogenous NLRP1 inflammasome in keratinocytes: MLN4924 and Bortezomib completely blocked mature IL-1β secretion and ASC oligomerization upon HRV14-3Cpro induction (FIG. 5E, lanes 5, 6 vs lane 4, FIG. 5F). It also stabilized the post-cleavage NLRP1 fragment, which was below the detection limit in untreated cells (FIG. 5E, lanes 5, 6 vs lane 4, black arrow). MLN4924 had a much less dramatic effect on Talabostat-induced IL-1β secretion in keratinocytes (FIG. 6B), suggesting the cullin^{ZER1/ZYG11B} system could not fully account for DPP8/9 inhibitor-mediated NLRP1 activation.

Given the known pleiotropic effects of MLN4924 on other cellular pathways, we used CRISPR/Cas9 to delete ZER1 and ZYG11B (termed ZZ-dKO) in 293T-ASC-GFP reporter cells. Clonal ZZ-dKO 293T-ASC-GFP cells demonstrated significant reductions in the percentage of ASC-GFP specks upon co-expression of wild-type NLRP1 and HRV14-3Cpro, whereas the UBR2 KO cells did not differ from Cas9 control 293T-ASC-GFP cells (FIG. 5G, FIG. 6C). As an additional control, the patient-derived gain-of-function NLRP1 mutation, p. M77T was examined. This mutation is by itself sufficient to cause the destabilization of the entire N-terminal fragment (FIG. 6A, lane 3 vs. lane 1), which is most probably due to disrupted NLRP1 folding [Zhong et al. Cell 167: 187-202.e17 (2016)] and independent of either UBR- or cullin^{ZER1/ZYG11B}-mediated N-degron pathways. Indeed, the percentage of ASC-GFP speck-forming cells caused by NLRP1^{M77T} did not differ in either ZZ-dKO or UBR2 KOs from control cells (FIG. 5G and FIG. 6C). Hence, cullin^{ZER1/ZYG11B} is specifically required for HRV14-3Cpro triggered human NLRP1 activation. The 3Cpro-cleaved NLRP1 fragment (a.a. 131-1213) was significantly stabilized in ZZ-dKO cells relative to control cells (FIG. 5H, lanes 6-8 vs. lane 5), but not in UBR2 KO cells (FIG. 6D, lanes 5-6 vs. lane 4). These results further demonstrate that the 3Cpro-cleaved NLRP1 autoproteolytic fragment (corresponding to a.a. 131-a.a.1212) is a substrate for the cullin^{ZER1/ZYG11B}-mediated N-terminal glycine degron pathway.

Taken together, these findings build a detailed model for 3Cpro-triggered human NLRP1 activation, which bears resemblance to, but differs significantly from the murine counterpart. Upon 3Cpro cleavage between Q130 and G131, the cleaved NLRP1 fragment (a.a. 131-1213) retains its auto-inhibitory activity despite the loss of the PYD domain. Subsequently, its exposed N-terminal glycine becomes recognized by the cullin^{ZER1/ZYG11B}-dependent N-glycine degron system. This constitutes an obligatory intermediate step for NLRP1 activation, as it routes the cleaved auto-inhibitory NLRP1 N-terminal fragment to the proteasome for ‘functional degradation’, while releasing the FIIND^{UPA}-CARD to self-oligomerize and engage ASC. This model is consistent with our initial observation that overexpressing the truncation mutant, NLRP1 a.a. 131-1474 did not lead to inflammasome activation (FIG. 5A). The most likely explanation is that the obligatory initiating methionine derived from the Kozak sequence in our overexpression construct masked the second glycine residue from being fully recognized by the cullin^{ZER1/ZYG11B} complex.

### Example 5

### NLRP1 is Required for Inflammasome Assembly Activation During HRV Infection

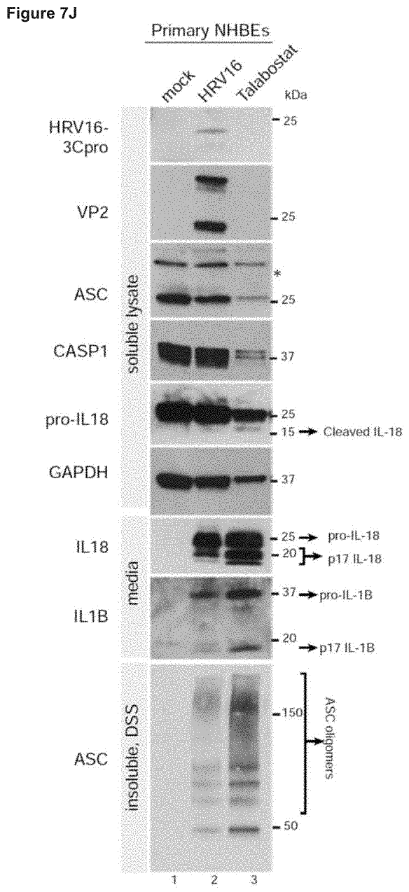

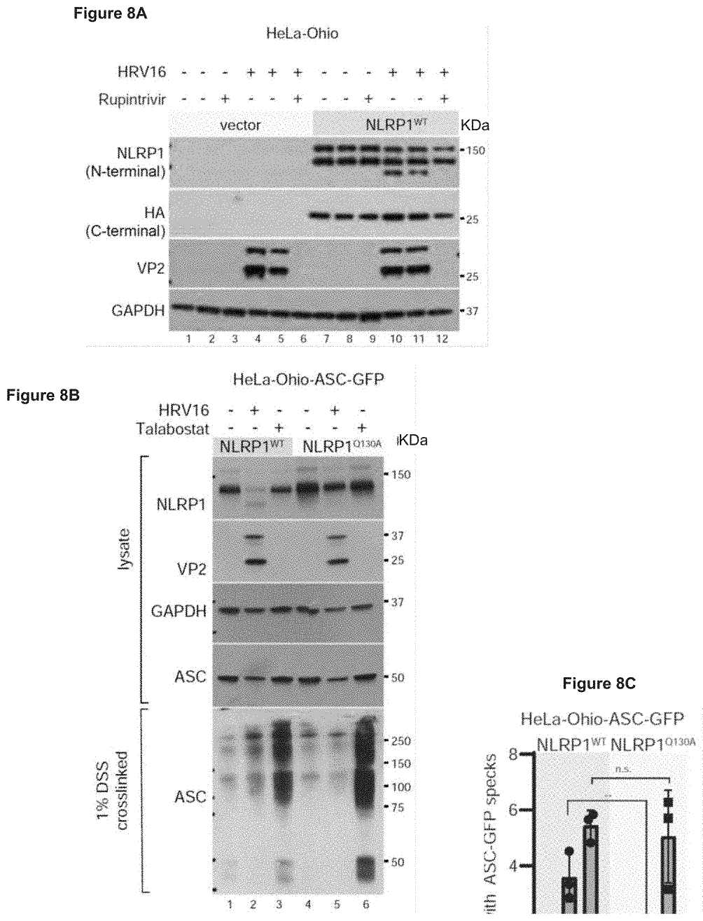

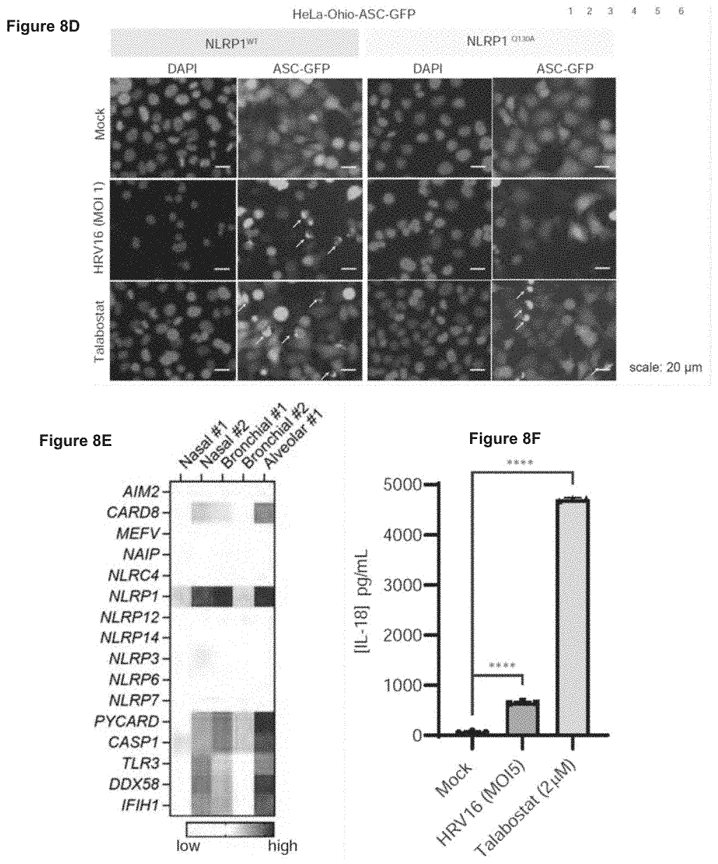

The effect of live HRV infection in disease-relevant human epithelial cell types was then examined. In HeLa-Ohio cells overexpressing NLRP1-HA, robust HRV16 viral replication was achieved 16 hours post inoculation (MOI=1) as detected by the accumulation of the viral capsid protein VP2 (FIG. 8A, lanes 4, 5, 10 and 11). This was accompanied by the appearance of the ˜120 kDa NLRP1 cleavage product in infected cells (FIG. 8A, lanes 10 and 11). The small molecule pan-HRV-3Cpro inhibitor, Rupintrivir completely abrogated NLRP1 cleavage (FIG. 8A, lane 12 vs. lanes 10 and 11). Using HeLa-Ohio cells that additionally expressed ASC-GFP, we next tested whether HRV16 infection could lead to NLRP1 inflammasome complex assembly. Even though HeLa-Ohio cells demonstrated less NLRP1 inflammasome activation than did keratinocytes or HEK293T cells, even for the positive control, Talabostat, HRV16 caused significant ASC-GFP oligomerization (FIG. 8B, lane 2 vs. lane 1) and speck formation (FIG. 8C-D). These effects were only observed in HeLa-Ohio-ASC-GFP cells expressing wild-type NLRP1, but not in cells expressing the cleavage-resistant NLRP1^{Q130A} mutant (FIG. 8B, lane 2 vs. lane 5, FIG. 8C-D. Therefore, live HRV16 infection causes 3Cpro-dependent NLRP1 cleavage and activates the reconstituted human NLRP1 inflammasome.

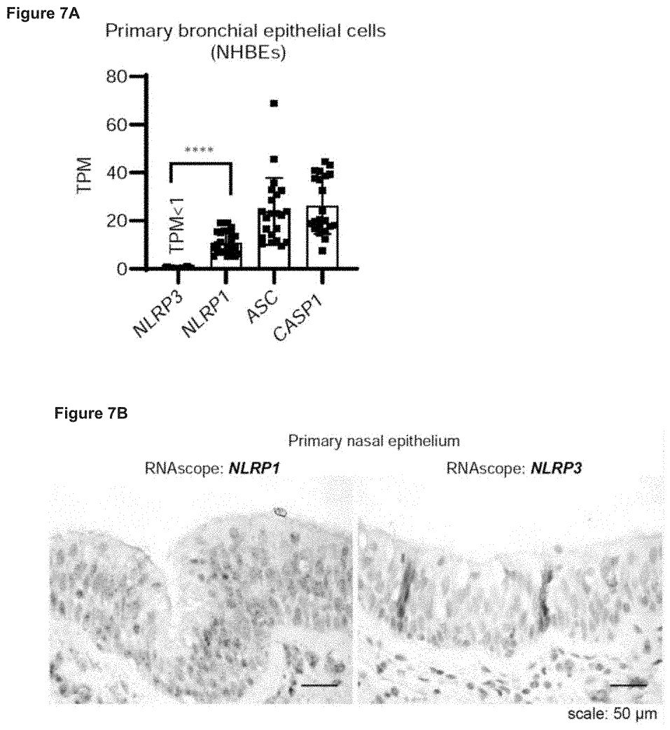

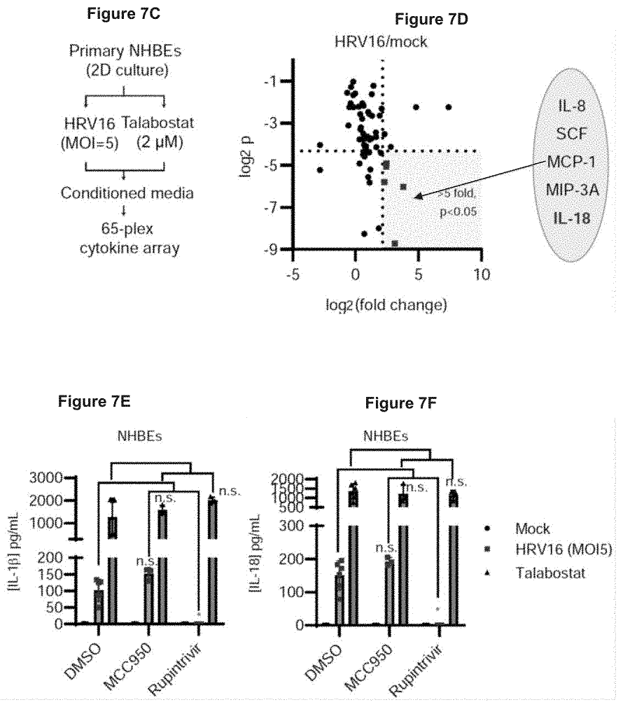

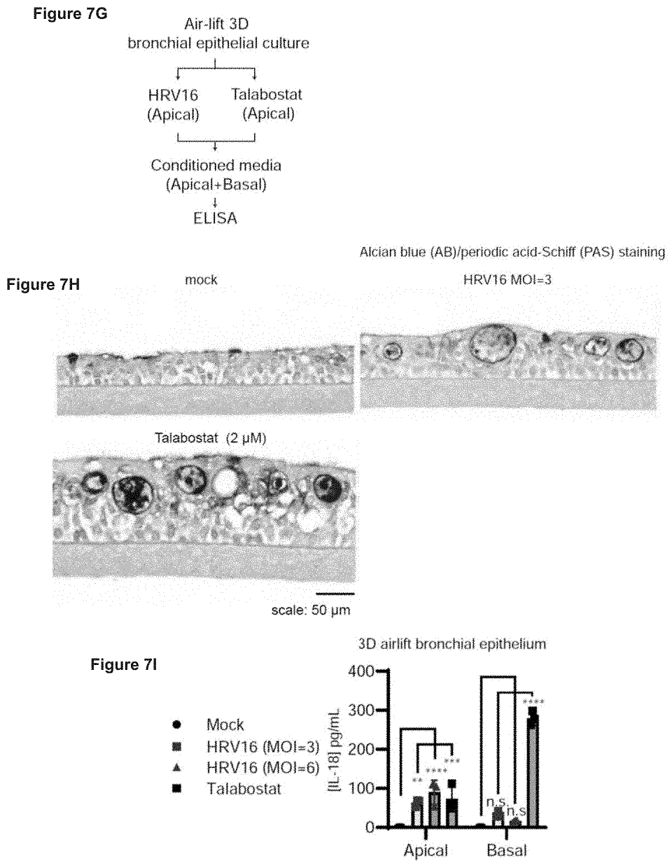

HRV is one of the most common human viral pathogens that cause respiratory tract infections. Human airway epithelial cells are known to endogenously express multiple dsRNA sensors such as TLR3, MDA5 and RIG-I, which all participate in antiviral defense. However, the repertoire of endogenous inflammasome components expressed in human airway epithelial cells have not been fully characterized. By examining published RNAseq datasets, we found that primary human airway epithelial cells [Landry et al., J. Infect. Dis. 217: 897-905 (2018); Mihaylova et al., Cell Rep. 24: 3000-3007.e3 (2018); Clark et al., PLoS One 10: e0115486 (2015); Hedstrom et al., Sci. Rep. 8:3502 (2018); Tian et al., BMC Genomics 16: 529 (2015)] express a very restricted repertoire of NLR sensors (FIG. 8E). Remarkably, other known inflammasome sensors, MEFV, NLRP3, NLRC5 and AIM2 were either lowly expressed or altogether absent (FIG. 8E). We confirmed that NLRP3 was not expressed in primary human bronchial epithelial cells across 21 replicates (TPM<1) (FIG. 7A). NLRP3 was also undetectable in primary human nasal epithelium by RNA in situ staining (FIG. 7B). The lack of NLRP3 inflammasome in NHBEs was recently confirmed by an independent study [Lee et al., Sci Immunol. 4 (2019), doi:10.1126/sciimmunoldotaau4643]. Although we cannot rule out that NLRP3 mRNA might be transcriptionally induced under specific conditions, these results demonstrate that NLRP1, but not NLRP3, is the predominant inflammasome sensor constitutively expressed in human airway epithelial cells. In addition to NLRP1, all airway epithelial cell types express endogenously PYCARD (ASC), CASP1 and IL-1 cytokines, IL1B and IL18 (FIG. 8E, FIG. 7A), suggesting that they are ‘primed’ to undergo NLRP1-mediated inflammasome activation.

We profiled the endogenous cytokine and chemokine response of primary human bronchial epithelial cells (NHBEs) to live HRV16 infection (FIG. 7C). Remarkably, IL-18, whose secretion strictly depends on inflammasome activation, was the most highly induced cytokine in both HRV16-infected and Talabostat-treated NHBEs (FIG. 7D). Subsequent ELISA experiments confirmed that IL-1β was also significantly secreted from HRV16-infected and Talabostat-treated NHBEs, but its levels were lower and more variable than those of IL-18 (FIG. 7E-F), likely because IL1B mRNA is subjected to transcriptional regulation. Similar results were obtained with primary nasal epithelial cells (FIG. 8F). In agreement with the lack of endogenous NLRP3 in NHBEs, neither IL-1β nor IL-18 secretion was affected by the NLRP3 inhibitor MCC950 after HRV16 infection or Talabostat treatment, whereas 3Cpro inhibitor Rupintrivir completely abrogated HRV16-induced, but not Talabostat-induced IL-1β and IL-18 secretion (FIG. 7E-F). We performed apical HRV16 infection of 3D air-lift culture of human bronchial epithelium to more closely mirror an incipient common cold infection of the human airway in vivo (FIG. 7G). Both HRV16 infection and Talabostat caused significant disruption of the epithelium, increased mucin production and goblet cell engorgement (FIG. 7H). In the case of HRV16 infection, this was accompanied by the secretion of IL-18 specifically in the apical culture supernatant where viral inoculation had taken place (FIG. 7I). In contrast, Talabostat led to IL-18 secretion in both the apical and basal media, likely due to its rapid diffusion through the culture (FIG. 7I).

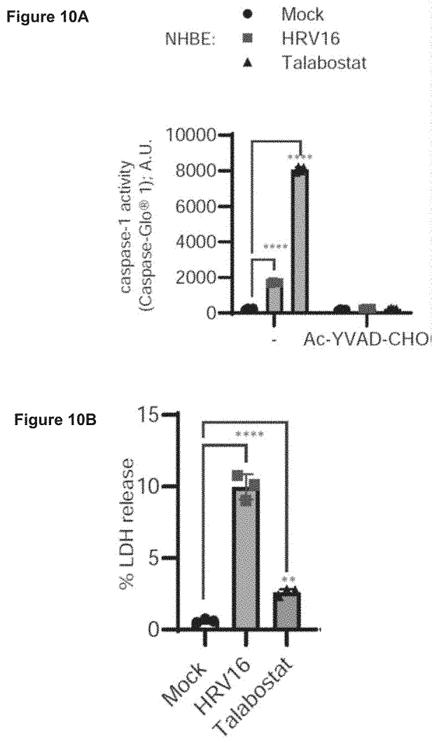

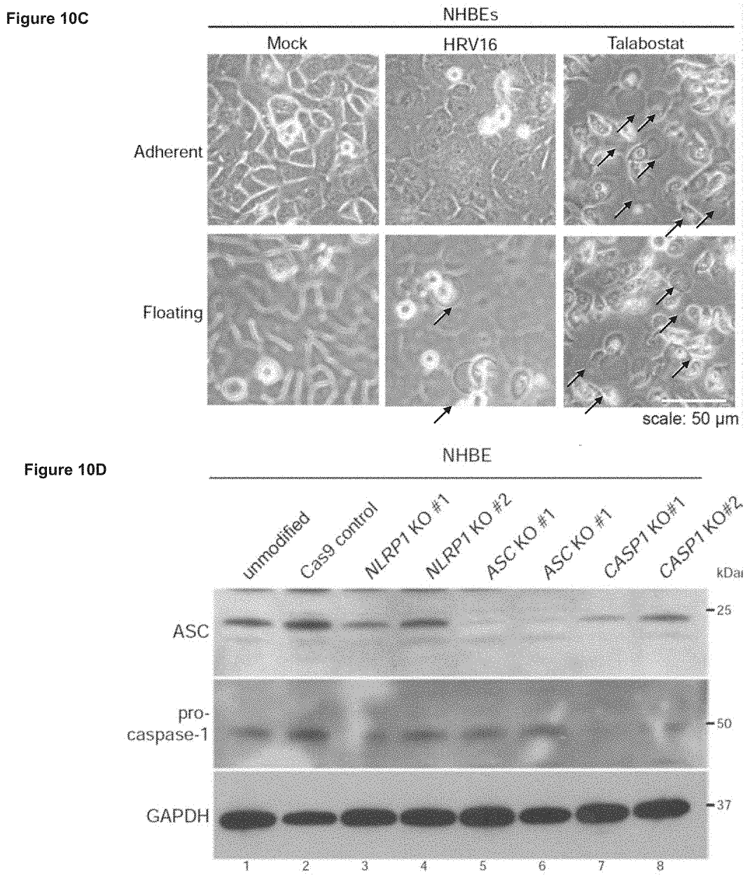

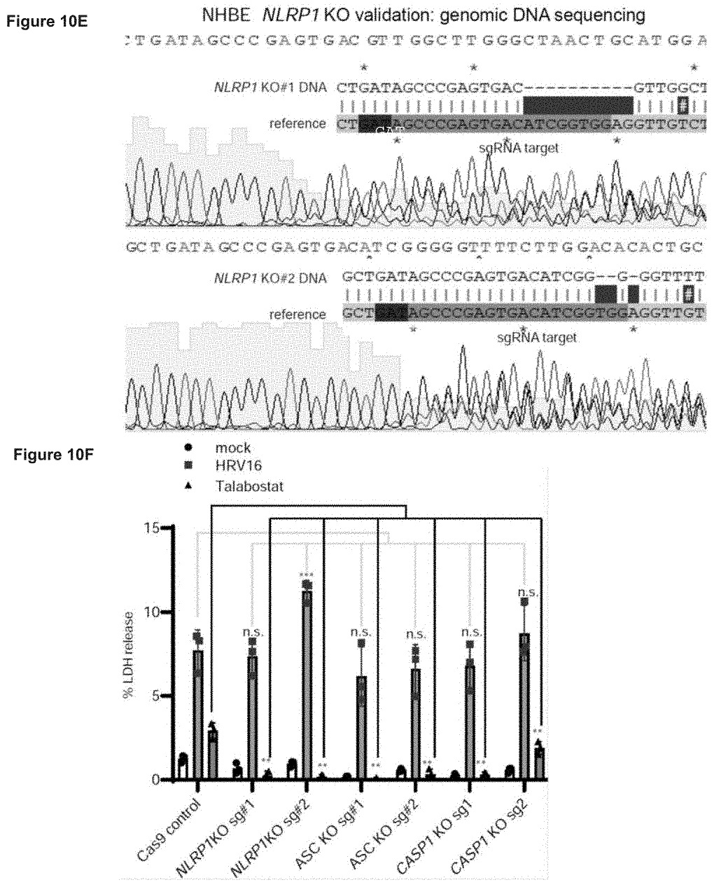

HRV16-infected NHBEs demonstrated cardinal features of inflammasome activation, including 1) proteolytic processing of IL-18 and IL-1β into their p17 mature forms (FIG. 7J, middle panels), 2) endogenous ASC oligomerization (FIG. 7J, lower panel), 3) caspase-1 activation (FIG. 10A), 4) release of intact LDH activity (FIG. 10B) and 5) characteristic membrane ‘ballooning’ (FIG. 10C, black arrows). Taken together, these results demonstrate that HRV16 infection activates the NLRP1, but not NLRP3 inflammasome pathway in primary human bronchial epithelial cells.

### Example 6

### HRV16 Infection Causes NLRP1 Activation Via Direct Cleavage and Requires the Cul^{ZER1/ZYG11B}-Mediated N-Glycine Degron Pathway

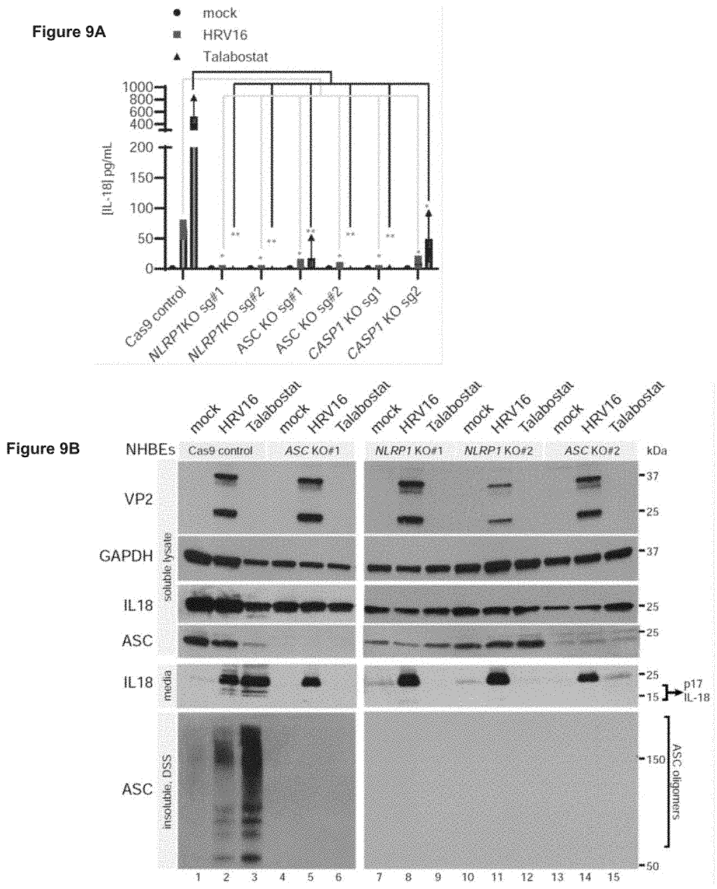

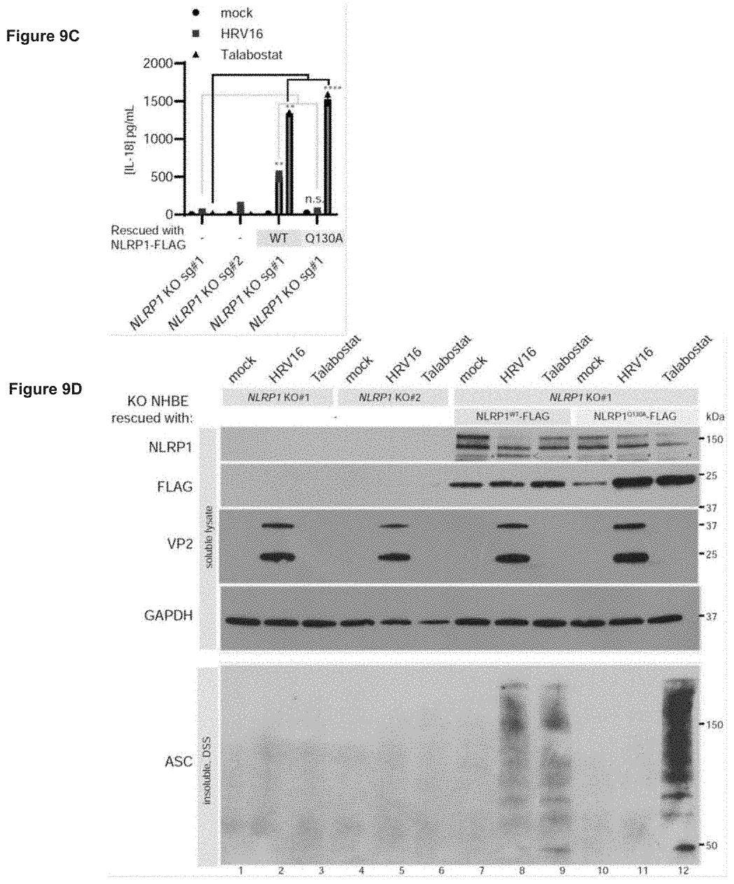

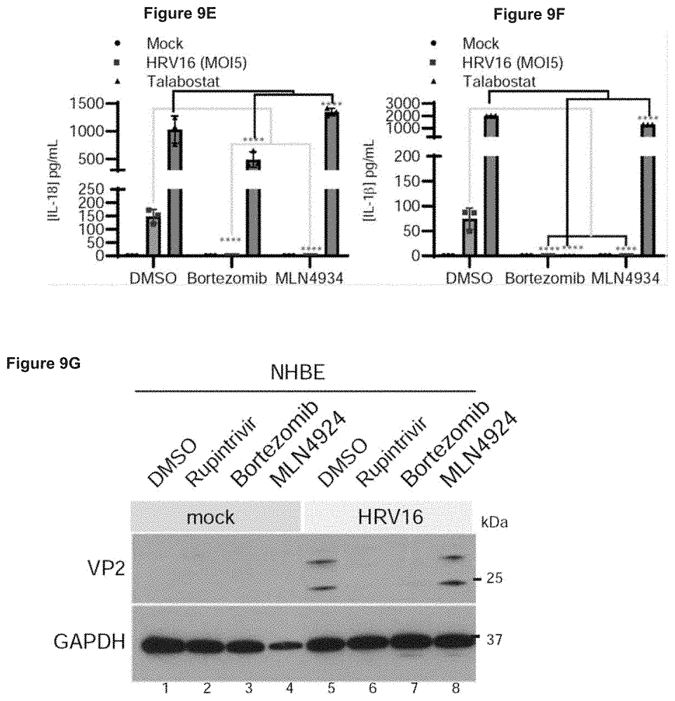

We sought to further validate whether endogenous NLRP1 is the obligate sensor for HRV-triggered inflammasome activation. CRISPR/Cas9-mediated deletion of NLRP1, its downstream adaptor ASC and pro-caspase-1 in primary NHBEs completely eliminated IL-18 cleavage caused by HRV16 infection or Talabostat treatment (FIG. 9A, FIG. 10D-E). Note that the endogenous NLRP1 in NHBEs was below the Western blot detection limit. Furthermore, HRV16-infected NLRP1 knockout NHBEs were unable to cleave IL-18 into its mature p17 form (FIG. 9B, lane 1-3 vs. 7-12), or assemble ASC into high-molecular weight oligomers (FIG. 9B, lanes 1-3 vs. lanes 7-12). This was not caused by a general defect in viral biogenesis, as the maturation of capsid protein VP2, which itself was dependent upon 3Cpro activity [Cordingley et al., J. Biol. Chem. 265: 9062-9065 (1990)], was not affected by NLRP1 or ASC KO (FIG. 9B, top panel). These results provide further support that NLRP1 is the primary inflammasome sensor for HRV infection in human NHBEs. HRV-triggered inflammasome activation likely occurs in parallel with other immune sensing pathways, as NLRP1, ASC and CASP1 KO NHBEs were still capable of producing other cytokines, such as IL-8, and undergoing cell death (FIG. 10F-G) upon HRV16 infection.

To confirm that HRV-induced NLRP1 activation requires the cleavage at a.a. Q130-G131, we infected NLRP1 KO NHBEs rescued with either FLAG-tagged wild-type NLRP1 or NLRP1^{Q130A} lentiviral constructs. As observed previously with keratinocytes (FIG. 3E-l), both wild-type NLRP1 and NLRP1^{Q130A} rescued Talabostat-dependent IL-18 secretion and ASC oligomerization in NLRP1 KO NHBEs (FIG. 9C, FIG. 9D, lane 9 and 12 vs. lane 3 and 6). However, only wild-type NLRP1, and not the uncleavable mutant Q130A, rescued HRV16-induced IL-18 secretion and ASC oligomerization (FIG. 9C, FIG. 9D, lane 8 vs. lane 11), despite similar levels of VP2 maturation. In addition, the 3Cpro cleavage fragment could only be detected in wild-type NLRP1-, but not NLRP1^{Q130A}-expressing cells (FIG. 9D, lane 8 vs. lane 11, top panel, black arrow. Note that this band occurs close to a degradation band present in all samples, asterisks). Taken together, these findings confirm that direct cleavage by 3Cpro is a necessary and sufficient step in HRV-induced NLRP1 activation in NHBEs. To the best of our knowledge, these findings identify 3Cpros as the first PAMP activator sensed by human NLRP1.

Whether HRV-dependent NLRP1 activation requires the N-terminal glycine degron pathway was investigated. MLN4924 inhibition of cullin^{ZER1/ZYG11B} and bortezomib inhibition of the proteasome completely blocked HRV16-dependent IL-1β and IL-18 secretion in NHBEs. (FIG. 9E-F). However, MLN4924 did not affect Talabostat-dependent IL-18 secretion and only had a modest effect on Talabostat-dependent IL-1β secretion (FIG. 9E-F). These findings confirm that both cullin^{ZER1/ZYG11B} and the proteasome are necessary for HRV-triggered NLRP1 inflammasome activation in NHBEs. As an important control, MLN4924, unlike Rupintrivir and Bortezomib, did not affect the accumulation of mature capsid protein VP2 (FIG. 9G), which is itself dependent on 3Cpro cleavage. Therefore, cullin^{ZER1/ZYG11B} is specifically required for HRV-induced NLRP1 activation, without affecting viral replication or 3Cpro activity.

### Summary

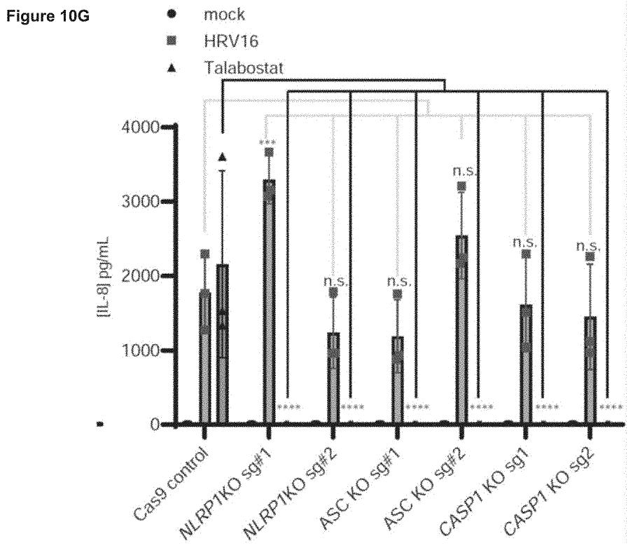

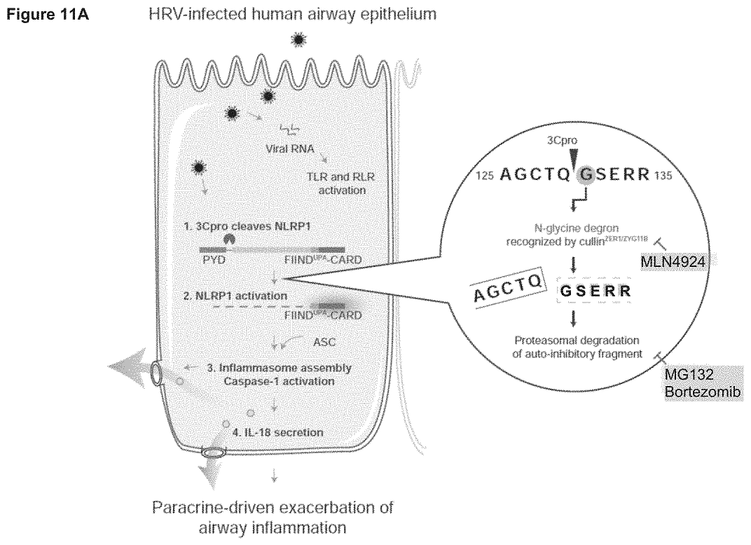

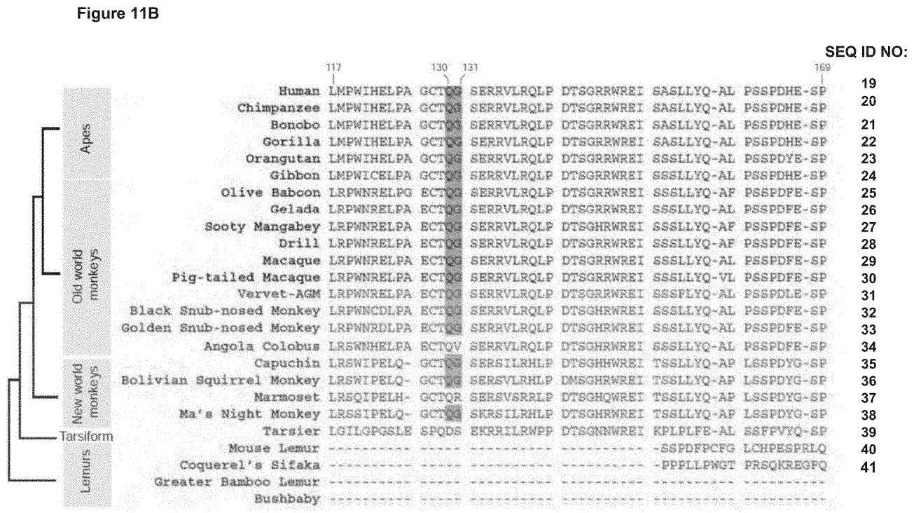

Enteroviral 3C proteases, such as HRV-3Cpro activate human inflammasome sensor NLRP1 via direct cleavage at a single site between a.a. Q130 and G131 (FIG. 11A). This discovery not only provides a unified mechanism for the NLRP1 inflammasome in humans and rodents [Chui et al., Science 364: 82-85 (2019); Sandstrom et al., Science 364 (2019) 10.1126/sciencedotaau1330; Xu et al., EMBO J. 38: (2019)], it also reveals an unexpected role for the recently described ‘N-terminal glycine degron’ pathway in human innate immunity. Mechanistically, 3Cpro-cleavage leaves a glycine residue (p. G131) on the NLRP1 fragment, which becomes a substrate of the cullin^{ZER1/ZYG11B} ‘N-glycine degron’ machinery and is subsequently degraded by the proteasome (FIG. 11A). This constitutes a key step in unleashing the NLRP1 FIIND^{UPA}-CARD fragment to fully assemble the inflammasome complex consisting of ASC and caspase-1. It is noteworthy that even though a single cleavage site mutation (Q130A) can completely block 3Cpros from activating the NLRP1 inflammasome, this mutation does not affect the DPP8/9 inhibitor Talabostat-triggered NLRP1 activation.

The identification of enteroviral 3Cpros as a PAMP trigger for human NLRP1 challenges the widely held notion that viral proteases largely serve to disable host immune sensing. It also sheds light on the evolutionary trajectory of NLRP1. The 3Cpro cleavage site arose in the common ancestor for simian primates (i.e. apes, new world and old world monkeys) and is absent in pro-simians such as tarsiers and lemurs (FIG. 11B). It is conceivable that the more recently evolved, 3Cpro-responsive NLRP1 allele has provided a selective survival advantage during the evolution of simian primates including humans, presumably to sense and mount an appropriate immune response against certain simian-tropic enteroviral pathogens. This does not preclude the possibility that human NLRP1 can detect other pathogen- or danger-derived signals besides 3Cpros.

The findings herein also establish human NLRP1 as a prominent viral sensor in the airway epithelium, which likely functions in parallel with other immune sensors such as TLRs and RLRs (FIG. 11B). As the inflammasome pathway does not require de novo protein synthesis, it is particularly well suited as a ‘fail-safe’ defence mechanism, when host transcription and translation have been shut off by viral virulence factors and can no longer make other antiviral molecules [Carrasco and Smith, Nature 264: 807-809 (1976)]. Using HRV as a model enterovirus, we demonstrate that NLRP1 and its cofactors are indispensable for HRV-triggered inflammasome activation in primary human bronchial epithelial cells. Based on these results, we propose that the NLRP1 inflammasome plays a critical role in both antiviral defence and pathological inflammation in the human airway epithelium. Respiratory viral infections, including HRVs, are well known risk factors for exacerbations for asthma and chronic obstructive pulmonary disease (COPD) [Jacobs et al., Clin. Microbiol. Rev. 26:135-162 (2013)]. Therefore, the NLRP1 inflammasome pathway is a potential therapeutic target to treat these diseases and their complications.

### REFERENCES

Agudelo D, Duringer A, et al., (2017) Marker-free coselection for CRISPR-driven genome editing in human cells. Nature Methods, 14: 615-620.
Boyden E. D., Dietrich W. F., Nalp1b controls mouse macrophage susceptibility to anthrax lethal toxin. Nat. Genet. 38, 240-244 (2006).
Buenz E. J., Howe C. L., Picornaviruses and cell death. Trends in Microbiology. 14 (2006), pp. 28-36.
Carrasco L., Smith A. E., Sodium ions and the shut-off of host cell protein synthesis by picornaviruses. Nature. 264, 807-809 (1976).
Chavarria-Smith J., Vance R. E., Direct proteolytic cleavage of NLRP1B is necessary and sufficient for inflammasome activation by anthrax lethal factor. PLoS Pathog. 9, e1003452 (2013).
Chavarria-Smith J., Mitchell P. S., Ho A. M., Daugherty M. D., Vance R. E., Functional and Evolutionary Analyses Identify Proteolysis as a General Mechanism for NLRP1 Inflammasome Activation. PLoS Pathog. 12, e1006052 (2016).
Chui A. J., Okondo M. C., Rao S. D., Gai K., Griswold A. R., Johnson D. C., Ball D. P., Taabazuing C. Y., Orth E. L., Vittimberga B. A., Bachovchin D. A., N-terminal degradation activates the NLRP1B inflammasome. Science. 364, 82-85 (2019).
Cirelli K. M., Gorfu G., Hassan M. A., Printz M., Crown D., Leppla S. H., Grigg M. E., Saeij J. P. J., Moayeri M., Inflammasome sensor NLRP1 controls rat macrophage susceptibility to Toxoplasma gondii. PLoS Pathog. 10, e1003927 (2014).
Clark J. G., Kim K.-H., Basom R. S., Gharib S. A., Plasticity of airway epithelial cell transcriptome in response to flagellin. PLoS One. 10, e0115486 (2015).
Cordingley M. G., Callahan P. L., Sardana V. V., Garsky V. M., Colonno R. J., Substrate requirements of human rhinovirus 3C protease for peptide cleavage in vitro. J. Biol. Chem. 265, 9062-9065 (1990).
Croft S. N., Walker E. J., Ghildyal R., Picornaviruses and Apoptosis: Subversion of Cell Death. mBio. 8 (2017), doi:10.1128/mbio.01009-17.
Croft S. N., Walker E. J., Ghildyal R., Human Rhinovirus 3C protease cleaves RIPK1, concurrent with caspase 8 activation. Sci. Rep. 8, 1569 (2018).
de Breyne S., Bonderoff J. M., Chumakov K. M., Lloyd R. E., Hellen C U. T., Cleavage of eukaryotic initiation factor eIF5B by enterovirus 3C proteases. Virology. 378, 118-122 (2008).
de Vasconcelos N. M., Vliegen G., Gonçalves A., De Hert E., Martin-Pérez R., Van Opdenbosch N., Jallapally A., Geiss-Friedlander R., Lambeir A.-M., Augustyns K., Van Der Veken P., De Meester I., Lamkanfi M., DPP8/DPP9 inhibition elicits canonical Nlrp1b inflammasome hallmarks in murine macrophages. Life Sci Alliance. 2 (2019), doi:10.26508/lsa.201900313.
D'Osualdo A., Weichenberger C. X., Wagner R. N., Godzik A., Wooley J., Reed J. C., CARD8 and NLRP1 undergo autoproteolytic processing through a ZU5-like domain. PLoS One. 6, e27396 (2011).
Drutman S. B., Haerynck F., Zhong F. L., Hum D., Hernandez N. J., Belkaya S., Rapaport F., de Jong S. J., Creytens D., Tavernier S. J., Bonte K., De Schepper S., van der Werff ten Bosch J., Lorenzo-Diaz L., Wullaert A., Bossuyt X., Orth G., Bonagura V. R., Beziat V., Abel L., Jouanguy E., Reversade B., Casanova J.-L., Homozygous NLRP1 gain-of-function mutation in siblings with a syndromic form of recurrent respiratory papillomatosis. Proceedings of the National Academy of Sciences. 116 (2019), pp. 19055-19063.
Ewald S. E., Chavarria-Smith J., Boothroyd J. C., NLRP1 is an inflammasome sensor for Toxoplasma gondii. Infect. Immun. 82, 460-468 (2014).
Finger J. N., Lich J. D., Dare L. C., Cook M. N., Brown K. K., Duraiswami C., Bertin J., Gough P. J., Autolytic proteolysis within the function to find domain (FIIND) is required for NLRP1 inflammasome activity. J. Biol. Chem. 287, 25030-25037 (2012).
Grandemange S., Sanchez E., Louis-Plence P., Mau-Them F. T., Bessis D., Coubes C., Frouin E., Seyger M., Girard M., Puechberty J., Costes V., Rodiere M., Carbasse A., Jeziorski E., Portales P., Sarrabay G., Mondain M., Jorgensen C., Apparailly F., Hoppenreijs E., Touitou I., Genevieve D., A new autoinflammatory and autoimmune syndrome associated with NLRP1 mutations: NAIAD (NLRP1-associated autoinflammation with arthritis and dyskeratosis). Annals of the Rheumatic Diseases. 76 (2017), pp. 1191-1198.
Hedström U., Hallgren O., Öberg L., DeMicco A., Vaarala O., Westergren-Thorsson G., Zhou X., Bronchial extracellular matrix from COPD patients induces altered gene expression in repopulated primary human bronchial epithelial cells. Sci. Rep. 8, 3502 (2018).
Hellmich K. A., Levinsohn J. L., Fattah R., Newman Z. L., Maier N., Sastalla I., Liu S., Leppla S. H., Moayeri M., Anthrax lethal factor cleaves mouse nlrp1b in both toxin-sensitive and toxin-resistant macrophages. PLoS One. 7, e49741 (2012).
Herlin T., Jorgensen S. E., Host C., Mitchell P. S., Christensen M. H., Laustsen M., Larsen D. A., Schmidt F. I., Christiansen M., Mogensen T. H., Autoinflammatory disease with corneal and mucosal dyskeratosis caused by a novel NLRP1 variant. Rheumatology (2019), doi:10.1093/rheumatology/kez612.
Jacobs S. E., Lamson D. M., St George K., Walsh T. J., Human rhinoviruses. Clin. Microbiol. Rev. 26, 135-162 (2013).
Jones J. D. G., Vance R. E., Dangl J. L., Intracellular innate immune surveillance devices in plants and animals. Science. 354 (2016), doi:10.1126/science.aaf6395.
Kanneganti T.-D., Lamkanfi M., Nuñez G., Intracellular NOD-like Receptors in Host Defense and Disease. Immunity. 27 (2007), pp. 549-559.
Landry M. L., Foxman E. F., Antiviral Response in the Nasopharynx Identifies Patients With Respiratory Virus Infection. J. Infect. Dis. 217, 897-905 (2018).
Latz E., The inflammasomes: mechanisms of activation and function. Current Opinion in Immunology. 22 (2010), pp. 28-33.
Lee S., Ishitsuka A., Noguchi M., Hirohama M., Fujiyasu Y., Petric P. P., Schwemmle M., Staeheli P., Nagata K., Kawaguchi A., Influenza restriction factor MxA functions as inflammasome sensor in the respiratory epithelium. Sci Immunol. 4 (2019), doi:10.1126/sciimmunol.aau4643.
Levandowski C. B., Mailloux C. M., Ferrara T. M., Gowan K., Ben S., Jin Y., McFann K. K., Holland P. J., Fain P. R., Dinarello C. A., Spritz R. A., NLRP1 haplotypes associated with vitiligo and autoimmunity increase interleukin-1 processing via the NLRP1 inflammasome. Proceedings of the National Academy of Sciences. 110 (2013), pp. 2952-2956.
Levinsohn J. L., Newman Z. L., Hellmich K. A., Fattah R., Getz M. A., Liu S., Sastalla I., Leppla S. H., Moayeri M., Anthrax lethal factor cleavage of Nlrp1 is required for activation of the inflammasome. PLoS Pathog. 8, e1002638 (2012).
Mamaï O., Boussofara L., Denguezli M., Escande-Beillard N., Kraeim W., Merriman B, Ben Charfeddine I., Stevanin G., Bouraoui S., Amara A., Mili A., Nouira R., H'mida D., Sriha B., Gribaa M., Saad A., Reversade B., Multiple self-healing palmoplantar carcinoma: a familial predisposition to skin cancer with primary palmoplantar and conjunctival lesions. J. Invest. Dermatol. 135, 304-308 (2015).
Matthews D. A., Smith W. W., Ferre R. A., Condon B., Budahazi G., Sisson W., Villafranca J. E., Janson C. A., McElroy H. E., Gribskov C. L., Structure of human rhinovirus 3C protease reveals a trypsin-like polypeptide fold, RNA-binding site, and means for cleaving precursor polyprotein. Cell. 77, 761-771 (1994).
Mihaylova V. T., Kong Y., Fedorova O., Sharma L., Dela Cruz C. S., Pyle A. M., Iwasaki A., Foxman E. F., Regional Differences in Airway Epithelial Cells Reveal Tradeoff between Defense against Oxidative Stress and Defense against Rhinovirus. Cell Rep. 24, 3000-3007.e3 (2018).
Mukherjee A., Morosky S. A., Delorme-Axford E., Dybdahl-Sissoko N., Steven Oberste M., Wang T., Coyne C. B., The Coxsackievirus B 3Cpro Protease Cleaves MAVS and TRIF to Attenuate Host Type I Interferon and Apoptotic Signaling. PLoS Pathogens. 7 (2011), p. e1001311.
Newman Z. L., Printz M. P., Liu S., Crown D., Breen L., Miller-Randolph S., Flodman P., Leppla S. H., Moayeri M., Susceptibility to anthrax lethal toxin-induced rat death is controlled by a single chromosome 10 locus that includes rNlrp1. PLoS Pathog. 6, e1000906 (2010).
Okondo M. C., Rao S. D., Taabazuing C. Y., Chui A. J., Poplawski S. E., Johnson D. C., Bachovchin D. A., Inhibition of Dpp8/9 Activates the Nlrp1b Inflammasome. Cell Chem Biol. 25, 262-267.e5 (2018).
Palmenberg A. C., Proteolytic processing of picornaviral polyprotein. Annu. Rev. Microbiol. 44, 603-623 (1990).
Piper S. C., Ferguson J., Kay L., Parker L. C., Sabroe I., Sleeman M. A., Briend E., Finch D. K., The role of interleukin-1 and interleukin-18 in pro-inflammatory and anti-viral responses to rhinovirus in primary bronchial epithelial cells. PLoS One. 8, e63365 (2013).
Sand J., Haertel E., Biedermann T., Contassot E., Reichmann E., French L. E., Werner S., Beer H.-D., Expression of inflammasome proteins and inflammasome activation occurs in human, but not in murine keratinocytes. Cell Death Dis. 9, 24 (2018).
Sandstrom A., Mitchell P. S., Goers L., Mu E. W., Lesser C. F., Vance R. E., Functional degradation: A mechanism of NLRP1 inflammasome activation by diverse pathogen enzymes. Science. 364 (2019), doi:10.1126/science.aau1330.
Shaw M. H., Reimer T., Kim Y.-G., Nuñez G., NOD-like receptors (NLRs): bona fide intracellular microbial sensors. Curr. Opin. Immunol. 20, 377-382 (2008).
Sui J., Li H., Fang Y., Liu Y., Li M., Zhong B., Yang F., Zou Q., Wu Y., NLRP1 gene polymorphism influences gene transcription and is a risk factor for rheumatoid arthritis in han chinese. Arthritis Rheum. 64, 647-654 (2012).
Tian B., Li X., Kalita M., Widen S. G., Yang. J., Bhavnani S. K., Dang B., Kudlicki A., Sinha M., Kong F., Wood T. G., Luxon B. A., Brasier A. R., Analysis of the TGFβ-induced program in primary airway epithelial cells shows essential role of NF-κB/RelA signaling network in type II epithelial mesenchymal transition. BMC Genomics. 16, 529 (2015).
Timms R. T., Zhang Z., Rhee D. Y., Harper J. W., Koren I, Elledge S. J., A glycine-specific N-degron pathway mediates the quality control of protein-myristoylation. Science. 365 (2019), doi:10.1126/science.aaw4912.
Varshavsky A., N-degron and C-degron pathways of protein degradation. Proc. Natl. Acad. Sci. U.S.A. 116, 358-366 (2019).
Walker E. J., Younessi P., Fulcher A. J., McCuaig R., Thomas B. J., Bardin P. G., Jans D. A., Ghildyal R., Rhinovirus 3C protease facilitates specific nucleoporin cleavage and mislocalisation of nuclear proteins in infected host cells. PLoS One. 8, e71316 (2013).
Wickliffe K. E., Leppla S. H., Moayeri M., Killing of macrophages by anthrax lethal toxin: involvement of the N-end rule pathway. Cell. Microbiol. 10, 1352-1362 (2008).
Xu H., Shi J., Gao H., Liu Y., Yang Z., Shao F., Dong N., The N-end rule ubiquitin ligase UBR2 mediates NLRP1B inflammasome activation by anthrax lethal toxin. EMBO J. 38, e101996 (2019).
Zell R., Picornaviridae—the ever-growing virus family. Arch. Virol. 163, 299-317 (2018).
Zhong F. L., Mamaï O., Sborgi L., Boussofara L., Hopkins R., Robinson K., Szeverényi I., Takeichi T., Balaji R., Lau A., Tye H., Roy K., Bonnard C., Ahl P. J., Jones L. A., Baker P. J., Lacina L., Otsuka A., Fournie P. R., Malecaze F., Lane E. B., Akiyama M., Kabashima K., Connolly J. E., Masters S. L., Soler V. J., Omar S. S., McGrath J. A., Nedelcu R., Gribaa M., Denguezli M., Saad A., Hiller S., Reversade B., Germline NLRP1 Mutations Cause Skin Inflammatory and Cancer Susceptibility Syndromes via Inflammasome Activation. Cell. 167, 187-202.e17 (2016).
Zhong F. L., Robinson K., Teo D. E. T., Tan K.-Y., Lim C., Harapas C. R., Yu C.-H., Xie W. H., Sobota R. M., Au V. B., Hopkins R., D'Osualdo A., Reed J. C., Connolly J. E., Masters S. L., Reversade B., Human DPP9 represses NLRP1 inflammasome and protects against autoinflammatory diseases via both peptidase activity and FIND domain binding. J. Biol. Chem. 293, 18864-18878 (2018).

1. A compound or composition comprising said compound for use in the prophylaxis or treatment of human airway inflammation and/or related complications triggered by Enterovirus 3C protease-cleaved NLRP1, wherein the compound or composition is an inhibitor of NLRP1 inflammasome activation.

2. The compound or composition comprising said compound of claim 1, wherein said 3C protease is from a virus species selected from the group comprising human enterovirus A (HEV-A), human enterovirus B (HEV-B), human enterovirus C (HEV-C), human enterovirus D (HEV-D), human rhinovirus A (HRV-A), human rhinovirus B (HRV-B), and human rhinovirus C (HRV-C).

3. The compound or composition comprising said compound of claim 1, wherein said compound inhibits N-glycine degron pathway ubiquitination and degradation of NLRP1 cleavage products.

4. The compound or composition comprising said compound of claim 3, wherein said compound inhibits cullin^{ZER1/ZYG11B}.

5. The compound or composition comprising said compound of claim 4, wherein said compound is an inhibitor of NEDD8-activating enzyme (NAE).

6. The compound or composition comprising said compound of claim 1, wherein said compound is selected from the group comprising:
MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof;
TAS4464, IUPAC name 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-, or hydrochloride salt thereof;
ZM223, IUPAC name N-[6-[[2-(4-aminophenyl)sulfanylacetyl]amino]-1,3-benzothiazol-2-yl]-4-(trifluoromethyl)benzamide;
MG132, IUPAC Name: benzyl N-[(2S)-4-methyl-1-[[(2S)-4-methyl-1-[[(2S)-4-methyl-1-oxopentan-2-yl]amino]-1-oxopentan-2-yl]amino]-1-oxopentan-2-yl]carbamate, and Bortezomib, IUPAC Name: [(1R)-3-methyl-1-[[(2S)-3-phenyl-2-(pyrazine-2-carbonyl amino)propanoyl]amino]butyl]boronic acid.

7. The compound or composition comprising said compound of claim 1, wherein said 3C protease is from a human rhinovirus.

8. The composition of claim 1, comprising pharmaceutically acceptable salts or solvates, or pharmaceutically functional derivatives of said inhibitor compound.

9. The composition of claim 1, comprising an inhibitor compound with a pharmaceutically-acceptable adjuvant, diluent or carrier.

10.-16. (canceled)

17. A method of prophylaxis or treatment of airway inflammation and/or related complications triggered by Enterovirus 3C protease-activated NLRP1, in a subject, comprising administering a therapeutically effective amount of a compound or composition of claim 1.

18. The method of claim 17, wherein said 3C protease is from a virus species selected from the group comprising human enterovirus A (HEV-A), human enterovirus B (HEV-B), human enterovirus C (HEV-C), human enterovirus D (HEV-D), human rhinovirus A (HRV-A), human rhinovirus B (HRV-B), and human rhinovirus C (HRV-C).

19. The method of claim 17, wherein said compound inhibits N-glycine degron pathway ubiquitination and degradation of NLRP1 cleavage products.

20. The method of claim 17, wherein said compound inhibits cullin^{ZER1/ZYG11B}.

21. The method of claim 17, wherein said compound is an inhibitor of NEDD8-activating enzyme (NAE).

22. The method of claim 17, wherein the compound is selected from the group comprising:
MLN4924, IUPAC name ((1S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof;
TAS4464, IUPAC name 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-, or hydrochloride salt thereof;
ZM223, IUPAC name N-[6-[[2-(4-aminophenyl)sulfanylacetyl]amino]-1,3-benzothiazol-2-yl]-4-(trifluoromethyl)benzamide;
MG132, IUPAC Name: benzyl N-[(2S)-4-methyl-1-[[(2S)-4-methyl-1-[[(2S)-4-methyl-1-oxopentan-2-yl]amino]-1-oxopentan-2-yl]amino]-1-oxopentan-2-yl]carbamate, and Bortezomib, IUPAC Name: [(1R)-3-methyl-1-[[(2S)-3-phenyl-2-(pyrazine-2-carbonyl amino)propanoyl]amino]butyl]boronic acid.

23. The method of claim 17, wherein a subject administered said prophylaxis or treatment will have reduced IL-1 secretion, ASC oligomerization and/or lytic cell death in the airway compared to an untreated subject.

24. The method of claim 17, wherein the compound is MLN4924, IUPAC name ((1 S,2S,4R)-4-(4-(((S)-2,3-dihydro-1H-inden-1-yl)amino)-7H-pyrrolo[2,3-d]pyrimidin-7-yl)-2-hydroxycyclopentyl)methyl sulfamate, or hydrochloride salt thereof.
